(19) United States
(12) Patent Application Publication
(10) Pub. No.: US 2003/0078212 A1
(43) Pub. Date: Apr. 24, 2003

(54) PHARMACEUTICAL COMPOSITIONS CONTAINING POLY(ADP-RIBOSE) GLYCOHYDROLASE INHIBITORS AND METHODS OF USING THE SAME

(76) Inventors: JIA-HE LI, COCKEYSVILLE, MD (US); JIE ZHANG, ELLICOTT CITY, MD (US)

Correspondence Address: GUILFORD PHARMACEUTICALS, INC., P.O. BOX 2903, MINNEAPOLIS, MN, 55402-0903, US

(21) Appl. No.: 09/182,645

(22) Filed: Oct. 30, 1998

(51) Int. Cl. A61K 31/70; A61K 31/47

(52) U.S. Cl. 514/22; 514/25

(57) ABSTRACT

The present invention relates to pharmaceutical compositions containing poly(ADP-ribose) glucohydrolase inhibitors, also known as PARG inhibitors, and methods of using the same for inhibiting or decreasing free radical induced cellular energy depletion, cell damage, or cell death. More particularly, the present invention relates to pharmaceutical compositions containing poly (ADP-ribose) glucohydrolase inhibitors such as glucose derivatives; lignin glycosides; hydrolysable tannins including gallotannins and ellagitannins; adenoside derivatives; acridine derivatives including 6,9-diamino-2-ethoxyacridine lactate monohydrate; tilorone analogs including tilorone R10.556, daunomycin or daunorubicin hydrochloride; ellipticine; proflavine; and other PARG inhibitors; and their method of use in treating or preventing diseases or conditions due to free radical induced cellular energy depletion and/or tissue damage resulting from cell damage or death due to necrosis, apoptosis, or combinations thereof.

### BACKGROUND OF THE INVENTION

1. Field of the Invention

The present invention relates to pharmaceutical compositions containing poly(ADP-ribose) glucohydrolase inhibitors, also known as PARG inhibitors, and methods of using the same for inhibiting or decreasing free radical induced cellular energy depletion, cell damage, or cell death. More particularly, the present invention relates to pharmaceutical compositions containing poly (ADP-ribose) glucohydrolase inhibitors such as glucose derivatives; lignin glycosides; hydrolysable tannins including gallotannins and ellagitannins; adenoside derivatives; acridine derivatives including 6,9-diamino-2-ethoxyacridine lactate monohydrate; tilorone analogs including tilorone R10.556, daunomycin or daunorubicin hydrochloride; ellipticine; proflavine; and other PARG inhibitors; and their method of use in treating or preventing diseases or conditions due to free radical induced cellular energy depletion and/or tissue damage resulting from cell damage or death due to necrosis, apoptosis, or combinations thereof.

2. Description of the Prior Art

A major focus of current biomedical research is on the mechanisms of cell death as new specific therapeutic agents which modulate these processes continue to be developed. Cell death is generally separated into two categories: apoptosis and necrosis. Apoptosis, commonly termed programmed cell death, has been particularly well characterized in development, while necrosis is more prominent as the initial response to overwhelming noxious insult. Programmed cell death is a genetically controlled process that follows physiologic stimuli in individual cells and typically involves ruffling of the cell membrane, nuclear and cytoplasmic condensation, intranucleosomal cleavage of DNA, and eventual phagocytosis of the cell without significant inflammation. Necrosis is a more rapid and severe process that occurs in groups of cells in response to pathologic injury. This mode of cell death is characterized by swelling of mitochondria and endoplasmic reticulum followed by a loss of membrane integrity and random destruction of DNA and other macromolecules culminating in substantial inflammatory response. Although the vast majority of cell death literature suggests that all instances of cell death can be classified as either apoptosis or necrosis, aspects of both mechanisms exist in a variety of cell death paradigms. One example is excitotoxicity following stroke and some neurodegenerative disorders in which neuronal death results at least in part from accumulation of high local concentrations of the excitatory neurotransmitter glutamate. While the immediate phase of cell death following hypoxia most closely resembles necrosis, propagation of the insult produces a secondary lesion with many features of classical apoptosis. To design rational therapeutic approaches to neuronal cell death in the future, researchers should probably consider individual disease paradigms as occupying unique positions somewhere on a continuum between the extremes of apoptosis and necrosis.

The DNA repair enzyme poly (ADP-ribose) polymerase (PARP) (EC 2.4.2.30), also known as poly (ADP-ribose) synthetase or poly (ADP-ribose) transferase (PADRT), has emerged as a major player along the continuum of cell death. Cleavage of PARP by caspase-3 is a defining characteristic of apoptosis, and PARP also plays a pivotal role in classical necrotic cell death as well. Nuclear PARP is selectively activated by DNA strand breaks to catalyze the addition of long, branched chains of poly (ADP-ribose) (PAR) from its substrate nicotinamide adenine dinucleotide (NAD) to a variety of nuclear proteins, most notably PARP itself. Massive DNA damage, such as that typically resulting from necrotic stimuli, elicits a major augmentation of PARP activity which rapidly depletes cellular levels of NAD. Depletion of NAD, an important co-enzyme in energy metabolism, results in lower ATP production. Furthermore, the cell consumes ATP in efforts to re-synthesize NAD, and this energy crisis culminates in cell death. The concept of PARP mediated cell death following excessive DNA damage is supported by a number of studies showing prevention of cell death by selective PARP inhibitors and protection in mice with targeted deletion of the PARP gene. Dramatic protection provided by PARP inhibition in a variety of animal models of disease may lead to new therapeutic entities.

Poly (ADP-ribosyl)ation is involved in a variety of physiologic events, such as chromate decondensation, DNA replication, DNA repair, gene expression, malignant transformation, cellular differentiation, and apoptosis. Nuclear PARP activity is abundant throughout the body, particularly in the brain, immune system and germ line cells. The PARP enzyme can be grouped into three major domains. A 46 kD N-terminal portion comprises the DNA binding domain which contains two zinc finger motifs and a nuclear localization signal. This region recognizes both double and single-stranded DNA breaks in a non-sequence dependent manner through the first and second zinc fingers, respectively. A 22 kD central automodification domain contains 15 highly conserved glutamate residues thought to be targets of self-poly(ADP-ribosyl)ation, and the 54 kD C-terminal region contains both the NAD binding site and the catalytic domain which synthesizes PAR.

Upon binding to breaks in DNA, PARP activity is increased as much as 500 fold as it catalyzes the transfer and polymerization of ADP-ribose units onto both itself and other nuclear proteins, including histones and DNA topoisomerases I and II. PARP itself is the main poly(ADP-ribosyl)ated protein in vivo. It is unclear how binding to DNA strand breaks by the N-terminal portion of PARP allosterically activates the catalytic domain, but initiation and subsequent elongation of the PAR polymer probably proceed by an intermolecular mechanism, such as protein dimerization. After initiation, PARP catalyzes elongation and branching reactions to synthesize highly branched and complex structures of over 200 ADP-ribose residues into a large homopolymer that is structurally similar to nucleic acids.

Poly(ADP-ribosyl)ation of proteins generally leads to their inhibition and can dissociate chromatin proteins from DNA. Poly(ADP-ribosyl)ation of histones, for example, decondenses chromatin structure, while subsequent degradation of the polymer restores chromatin to its condensed form. Relaxation of chromatin may mediate DNA events at damaged sites as well as origins of replication and transcription initiation sites. One hypothesis is that PARP helps maintain chromosomal integrity by protecting broken DNA from inappropriate homologous recombination. The binding of PARP to DNA ends could preclude their association with genetic recombination machinery, and negatively charged PAR could electrostatically repel other DNA molecules. Auto-poly(ADP-ribosyl)ation inactivates PARP through electrostatic repulsion between negatively charged enzyme-bound ADP-ribose polymers and DNA, and release of PARP from DNA allows access of DNA repair enzymes to the lesion (FIG. 1).

PAR that is synthesized in response to massive DNA damage has a short half-life close to one minute as it is rapidly hydrolyzed at ribose-ribose bonds and converted to free ADP-ribose by the enzyme poly(ADP-ribose)glycohydrolase (PARG). The rapid response of PARG to PAR synthesis indicates that PAR degradation is also an important nuclear response to DNA damage. Accordingly, the results shown herein suggest that the conversion of PAR to free ADP-ribose by PARG can further promote PARP activity by providing additional substrate (ADP-ribose) for PARP and additional targets for poly(ADP-ribosyl)ation (sites where PARG has cleaved away ADP-ribose units). The activation of PARG thereby promotes the PARP-induced depletion of cellular energy, increased cell damage and cell death associated with the diseases and disorders linked to PARP activity as described herein. Although this is believed to be the mode of action, other mechanisms of action may be responsible for, or contribute to, the usefulness of PARG inhibitors described herein including methods for treating or preventing the disorders or diseases described herein. Recently, bovine cDNA encoding PARG was cloned. While PARG is approximately 13-50 fold less abundant than PARP, its specific activity is about 50 to 70 fold higher. The cell expends considerable energy in rapid synthesis and degradation of PAR polymer, suggesting that like PARP, PARG might be a useful target for pharmacologic intervention.

PARP activation is an extremely sensitive indicator of DNA damage, appearing much earlier and exceeding in magnitude the augmentation of DNA nicks monitored by terminal-deoxynucleotidyl transferase. Since a large array of nuclear proteins are covalently modified with PAR immediately following DNA breakage, poly(ADP-ribosyl)ation is considered a major player in cellular response to DNA damage. Mutant cell lines with reduced expression of PARP exhibit compromised DNA repair, and PARP inhibitors render cells hypersensitive to DNA-damaging agents. Furthermore, depletion of PARP through expression of antisense PARP mRNA inhibits strand break rejoining in damaged DNA.

The development by two independent groups of mice with targeted deletion of PARP has provided an opportunity to more definitively evaluate the role of this enzyme in DNA repair. Wang et al. have generated knockout mice by disrupting exon 2, while Menissier de Murcia at al. have interrupted exon 4. Both strains of mutant mice are healthy and fertile, and fibroblasts from the Wang PARP −/− mice show normal DNA repair following DNA damage by UV irradiation or alkylating agents, representing respectively the efficiency of nucleotide excision repair and base excision repair systems. While proliferation of PARP −/− primary fibroblasts or in vivo thymocytes following γ-irradiation is somewhat impaired, the only significant defect observed by Wang et al. in their knockout mice is increased susceptibility to epidermal hyperplasia. Wang et al. have proposed that a lack of PARP activity in keratinocytes may prevent elimination of cells that contain large amounts of damaged DNA, thus rendering these cells more susceptible to hyperproliferation. No epidermal diseases were observed in the other strain of PARP −/− mice, however, suggesting that epidermal hyperplasia could be secondary to genetic background.

PARP −/− mice from the Menissier de Murcia group, on the other hand, do show abnormal responses to DNA damage. These PARP −/− cells are extremely sensitive to apoptosis following treatment with the alkylating agent N-methyl-N-nitrosourea (MNU). They also exhibit elevated p53 accumulation, probably due to a lack of or delay in DNA repair. This indicates that in these mice, lack of PARP accelerates p53 response to DNA damage. This is in contrast to what has been observed with the Wang PARP −/− mice, whose fibroblasts manifest the sane decrease in p53 as wild type DNA damage. Others have demonstrated that poly(ADP-ribosyl)ation serves a modulatory role in p53 signaling in wild type cells. It appears that while p53 levels may be partly determined by PARP activity, p53 activation is largely independent of PARP. The rate of sister chromatid exchange in the de Murcia PARP −/− mice is 4-5 times higher than the rate in WT mice at both basal levels and following DNA damage. This confirms earlier in situ results with a dominant-negative mutant of human PARP which suggested a role of PARP in limiting sister chromatid exchange following DNA damage. Both types of knockout mice die more rapidly than wild type mice following treatment with the methylating agent MNU or whole body γ-irradiation. The rapid activation of PARG in response to PAR synthesis and PARP activation indicates that PAR degradation via PARG should promote the disorders and diseases associated with PARP activity. Accordingly, PARG inhibitors should be useful in down-regulating PARP by decreasing substrate and targets for PARP activity, and thus PARG inhibitors are useful for treating disorders and diseases associated with PARP activity especially those disorders and diseases suggested herein. PARG inhibitors should be useful for any methods and therapies where the use of PARP inhibitors are useful.

It has been reported that PARP activation plays a key role in both NMDA- and NO-induced neurotoxicity, as shown by the use of PARP inhibitors to prevent such toxicity in cortical cultures in proportion to their potencies as inhibitors of this enzyme (Zhang et al., “Nitric Oxide Activation of Poly(ADP-Ribose) Synthetase in Neurotoxicity”, Science, 263:687-89 (1994)); and in hippocampal slices (Wallis et al., “Neuroprotection Against Nitric Oxide Injury with Inhibitors of ADP-Ribosylation”, NeuroReport, 5:3, 245-48 (1993)). The potential role of PARP inhibitors in treating neurodegenerative diseases and head trauma has thus been known. Research, however, continues to pinpoint the exact mechanisms of their salutary effect in cerebral ischemia, (Endres et al., “Ischemic Brain Injury is Mediated by the Activation of Poly(ADP-Ribose)Polymerase”, J. Cereb. Blood Flow Metabol., 17:1143-51 (1997)) and in traumatic brain injury (Wallis et al., “Traumatic Neuroprotection with Inhibitors of Nitric Oxide and ADP-Ribosylation, Brain Res., 710:169-77 (1996)). PARG inhibitors should influence PARP-associated NMDA- and NO-induced neurotoxicity by downregulating PARP activity and thus PARG inhibitors are useful for treating neurodegenerative diseases, head trauma, and cerebral ischemia.

It has been demonstrated that single injections of PARP inhibitors have reduced the infarct size caused by ischemia and reperfusion of the heart or skeletal muscle in rabbits. In these studies, a single injection of the PARP inhibitor, 3-amino-benzamide (10 mg/kg), either one minute before occlusion or one minute before reperfusion, caused similar reductions in infarct size in the heart (32-42%). Another PARP inhibitor, 1,5-dihydroxyisoquinoline (1 mg/kg), reduced infarct size by a comparable degree (38-48%). Thiemermann et al., “Inhibition of the Activity of Poly(ADP Ribose) Synthetase Reduces Ischemia-Reperfusion Injury in the Heart and Skeletal Muscle”, Proc. Natl. Acad. Sci. USA, 94:679-83 (1997). This finding has suggested that PARP inhibitors might be able to salvage previously ischemic heart or skeletal muscle tissue. Likewise, PARG inhibitors should influence PARP-associated ischemic heart or skeletal muscle tissue damage by downregulating PARP activity and thus PARG inhibitors are useful for salvaging previously ischemic heart or skeletal muscle tissue.

PARP activation has also been shown to provide an index of damage following neurotoxic insults by glutamate (via NMDA receptor stimulation), reactive oxygen intermediates, amyloid β-protein, n-methyl-4-phenyl-1,2,3,6-tetrahydropyridine (MPTP) and its active metabolite N-methyl-4-phenylpyridine (MPP⁺), which participate in pathological conditions such as stroke, Alzheimer's disease and Parkinson's disease. Zhang et al., “Poly(ADP-Ribose) Synthetase Activation: An Early Indicator of Neurotoxic DNA Damage”, J. Neurochem., 65:3, 1411-14 (1995). Other studies have continued to explore the role of PARP activation in cerebellar granule cells in vitro and in MPTP neurotoxicity. Cosi et al., “Poly(ADP-Ribose) Polymerase (PARP) Revisited. A New Role for an Old Enzyme: PARP Involvement in Neurodegeneration and PARP Inhibitors as Possible Neuroprotective Agents”, Ann. N.Y. Acad. Sci., 825:366-79 (1997); and Cosi et al., “Poly(ADP-Ribose) Polymerase Inhibitors Protect Against MPTP-induced Depletions of Striatal Dopamine and Cortical Noradrenaline in C57B1/6 Mice”, Brain Res., 729:264-69 (1996). PARG inhibitors should influence PARP-associated neurotoxic insults by glutamate (via NMDA receptor stimulation), reactive oxygen intermediates, amyloid β-protein, n-methyl-4-phenyl-1,2,3,6-tetrahydropyridine (MPTP) and its active metabolite N-methyl-4-phenylpyridine (MPP⁺), which participate in pathological conditions such as stroke, Alzheimer's disease and Parkinson's disease by downregulating PARP activity and thus PARG inhibitors are useful for treating or preventing such pathological conditions.

Neural damage following stroke and other neurodegenerative processes is thought to result from a massive release of the excitatory neurotransmitter glutamate, which acts upon the N-methyl-D-aspartate (NMDA) receptors and other subtype receptors. Glutamate serves as the predominate excitatory neurotransmitter in the central nervous system (CNS). Neurons release glutamate in great quantities when they are deprived of oxygen, as may occur during an ischemic brain insult such as a stroke or heart attack. This excess release of glutamate in turn causes over-stimulation (excitotoxicity) of N-methyl-D-aspartate (NMDA), AMPA, Kainate and MGR receptors. When glutamate binds to these receptors, ion channels in the receptors open, permitting flows of ions across their cell membranes, e.g., Ca²⁺ and Na⁺ into the cells and K⁺ out of the cells. These flows of ions, especially the influx of Ca²⁺, cause overstimulation of the neurons. The over-stimulated neurons secrete more glutamate, creating a feedback loop or domino effect which ultimately results in cell damage or death via the production of proteases, lipases and free radicals. Excessive activation of glutamate receptors has been implicated in various neurological diseases and conditions including epilepsy, stroke, Alzheimer's disease, Parkinson's disease, Amyotrophic Lateral Sclerosis (ALS), Huntington's disease, schizophrenia, chronic pain, ischemia and neuronal loss following hypoxia, hypoglycemia, ischemia, trauma, and nervous insult. Recent studies have also advanced a glutamatergic basis for compulsive disorders, particularly drug dependence. Evidence includes findings in many animal species, as well as, in cerebral cortical cultures treated with glutamate or NMDA, that glutamate receptor antagonists block neural damage following vascular stroke. Dawson et al., “Protection of the Brain from Ischemia”, Cerebrovascular Disease, 319-25 (H. Hunt Batjer ed., 1997). Attempts to prevent excitotoxicity by blocking NMDA, AMPA, Kainate and MGR receptors have proven difficult because each receptor has multiple sites to which glutamate may bind. Many of the compositions that are effective in blocking the receptors are also toxic to animals. As such, there is no known effective treatment for glutamate abnormalities.

The stimulation of NMDA receptors, in turn, activates the enzyme neuronal nitric oxide synthase (NNOS), which causes the formation of nitric oxide (NO), which more directly mediates neurotoxicity. Protection against NMDA neurotoxicity has occurred following treatment with NOS inhibitors. See Dawson et al., “Nitric Oxide Mediates Glutamate Neurotoxicity in Primary Cortical Cultures”, Proc. Natl. Acad. Sci. USA, 88:6368-71 (1991); and Dawson et al., “Mechanisms of Nitric Oxide-mediated Neurotoxicity in Primary Brain Cultures”, J. Neurosci., 13:6, 2651-61 (1993). Protection against NMDA neurotoxicity can also occur in cortical cultures from mice with targeted disruption of NNOS. See Dawson et al., “Resistance to Neurotoxicity in Cortical Cultures from Neuronal Nitric Oxide Synthase-Deficient Mice”, J. Neurosci., 16:8, 2479-87 (1996).

It is known that neural damage following vascular stroke is markedly diminished in animals treated with NOS inhibitors or in mice with NNOS gene disruption. Iadecola, “Bright and Dark Sides of Nitric Oxide in Ischemic Brain Injury”, Trends Neurosci., 20:3, 132-39 (1997); and Huang et al., “Effects of Cerebral Ischemia in Mice Deficient in Neuronal Nitric Oxide Synthase”, Science, 265:1883-85 (1994). See also, Beckman et al., “Pathological Implications of Nitric Oxide, Superoxide and Peroxynitrite Formation”, Biochem. Soc. Trans., 21:330-34 (1993). Either NO or peroxynitrite can cause DNA damage, which activates PARP. Further support for this is provided in Szabó et al., “DNA Strand Breakage, Activation of Poly(ADP-Ribose) Synthetase, and Cellular Energy Depletion are Involved in the Cytotoxicity in Macrophages and Smooth Muscle Cells Exposed to Peroxynitrite”, Proc. Natl. Acad. Sci. USA, 93:1753-58 (1996).

Zhang et al., U.S. Pat. No. 5,587,384 issued Dec. 24, 1996, discusses the use of certain PARP inhibitors, such as benzamide and 1,5-dihydroxy-isoquinoline, to prevent NMDA-mediated neurotoxicity and, thus, treat stroke, Alzheimer's disease, Parkinson's disease and Huntington's disease. However, it has now been discovered that Zhang et al. may have been in error in classifying neurotoxicity as NMDA-mediated neurotoxicity. Rather, it may have been more appropriate to classify the in vivo neurotoxicity present as glutamate neurotoxicity. See Zhang et al. “Nitric Oxide Activation of Poly(ADP-Ribose) Synthetase in Neurotoxicity”, Science, 263:687-89 (1994). See also, Cosi et al., Poly(ADP-Ribose)Polymerase Inhibitors Protect Against MPTP-induced Depletions of Striatal Dopamine and Cortical Noradrenaline in C57B1/6 Mice”, Brain Res., 729:264-69 (1996). PARG inhibitors should influence PARP-associated glutamate neurotoxicity by downregulating PARP activity and thus PARG inhibitors are useful for treating or preventing the glutamate neurotoxicity associated disorders and diseases discussed herein.

It is also known that PARP inhibitors affect DNA repair generally. Cristovao et al., “Effect of a Poly(ADP-Ribose) Polymerase Inhibitor on DNA Breakage and Cytotoxicity Induced by Hydrogen Peroxide and γ-Radiation,” Terato., Carcino., and Muta., 16:219-27 (1996), discusses the effect of hydrogen peroxide and γ-radiation on DNA strand breaks in the presence of and in the absence of 3-aminobenzamide, a potent inhibitor of PARP. Cristovao et al. observed a PARP-dependent recovery of DNA strand breaks in leukocytes treated with hydrogen peroxide. PARG inhibitors should influence PARP-associated DNA repair by downregulating PARP activity and thus PARG inhibitors are useful for treating or preventing the disorders and diseases discussed herein associated with DNA damage and DNA repair.

PARP inhibitors have been reported to be effective in radiosensitizing hypoxic tumor cells and effective in preventing tumor cells from recovering from potentially lethal damage of DNA after radiation therapy, presumably by their ability to prevent DNA repair. See U.S. Pat. Nos. 5,032,617; 5,215,738; and 5,041,653. PARG inhibitors should influence PARP-associated radiosensitization by downregulating PARP activity and thus PARG inhibitors are useful as radiosensitizers or agents associated with radiosensitization.

Evidence also exists that PARP inhibitors are useful for treating inflammatory bowel disorders. Salzman et al., “Role of Peroxynitrite and Poly(ADP-Ribose)Synthase Activation Experimental Colitis,” Japanese J. Pharm., 75, Supp. I:15 (1997), discusses the ability of PARP inhibitors to prevent or treat colitis. Colitis was induced in rats by intraluminal administration of the hapten trinitrobenzene sulfonic acid in 50% ethanol. Treated rats received 3-aminobenzamide, a specific inhibitor of PARP activity. Inhibition of PARP activity reduced the inflammatory response and restored the morphology and the energetic status of the distal colon. See also, Southan et al., “Spontaneous Rearrangement of Aminoalkylithioureas into Mercaptoalkylguanidines, a Novel Class of Nitric Oxide Synthase Inhibitors with Selectivity Towards the Inducible Isoform”, Br. J. Pharm., 117:619-32 (1996); and Szabó et al., “Mercaptoethylguanidine and Guanidine Inhibitors of Nitric Oxide Synthase React with Peroxynitrite and Protect Against Peroxynitrite-induced Oxidative Damage”, J. Biol. Chem., 272:9030-36 (1997). PARG inhibitors should influence PARP-associated colitis by downregulating PARP activity and thus PARG inhibitors are useful for treating or preventing the symptoms, disorders or diseases associated with colitis as discussed herein.

Evidence also exists that PARP inhibitors are useful for treating arthritis. Szabó et al., “Protective Effects of an Inhibitor of Poly(ADP-Ribose)Synthetase in Collagen-Induced Arthritis,” Japanese J. Pharm., 75, Supp. I:102 (1997), discusses the ability of PARP inhibitors to prevent or treat collagen-induced arthritis. See also Szabó et al., “DNA Strand Breakage, Activation of Poly(ADP-Ribose)Synthetase, and Cellular Energy Depletion are Involved in the Cytotoxicity in Macrophages and Smooth Muscle Cells Exposed to Peroxynitrite,” Proc. Natl. Acad. Sci. USA, 93:1753-58 (March 1996); Bauer et al., “Modification of Growth Related Enzymatic Pathways and Apparent Loss of Tumorigenicity of a ras-transformed Bovine Endothelial Cell Line by Treatment with 5-Iodo-6-amino-1,2-benzopyrone (INH₂BP)”, Intl. J. Oncol., 8:239-52 (1996); and Hughes et al., “Induction of T Helper Cell Hyporesponsiveness in an Experimental Model of Autoimmunity by Using Nonmitogenic Anti-CD3 Monoclonal Antibody”, J. Immuno., 153:3319-25 (1994). PARG inhibitors should influence PARP-associated arthritis by downregulating PARP activity and thus PARG inhibitors are useful for treating or preventing arthritis and the arthritis associated disorders and diseases discussed herein.

Further, PARP inhibitors appear to be useful for treating diabetes. Heller et al., “Inactivation of the Poly(ADP-Ribose)Polymerase Gene Affects Oxygen Radical and Nitric Oxide Toxicity in Islet Cells,” J. Biol. Chem., 270:19, 11176-80 (May 1995), discusses the tendency of PARP to deplete cellular NAD+ and induce the death of insulin-producing islet cells. Heller et al. used cells from mice with inactivated PARP genes and found that these mutant cells did not show NAD+ depletion after exposure to DNA-damaging radicals. The mutant cells were also found to be more resistant to the toxicity of NO. PARG inhibitors should influence PARP-associated diabetes by downregulating PARP activity and thus PARG inhibitors are useful for treating or preventing diabetes and diabetes associated disorders and diseases discussed herein.

Further still, PARP inhibitors have been shown to be useful for treating endotoxic shock or septic shock. Zingarelli et al., “Protective Effects of Nicotinamide Against Nitric Oxide-Mediated Delayed Vascular Failure in Endotoxic Shock: Potential Involvement of PolyADP Ribosyl Synthetase,” Shock, 5:258-64 (1996), suggests that inhibition of the DNA repair cycle triggered by poly(ADP ribose) synthetase has protective effects against vascular failure in endotoxic shock. Zingarelli et al. found that nicotinamide protects against delayed, NO-mediated vascular failure in endotoxic shock. Zingarelli et al. also found that the actions of nicotinamide may be related to inhibition of the NO-mediated activation of the energy-consuming DNA repair cycle, triggered by poly(ADP ribose) synthetase. See also, Cuzzocrea, “Role of Peroxynitrite and Activation of Poly(ADP-Ribose) Synthetase in the Vascular Failure Induced by Zymosan-activated Plasma,” Brit. J. Pharm., 122:493-503 (1997). PARG inhibitors should influence PARP-associated endotoxic shock or septic shock by downregulating PARP activity and thus PARG inhibitors are useful for treating or preventing endotoxic shock or septic shock and associated disorders or diseases as discussed herein.

Yet another known use for PARP inhibitors is treating cancer. Suto et al., “Dihydroisoquinolinones: The Design and Synthesis of a New Series of Potent Inhibitors of Poly(ADP-Ribose) Polymerase”, Anticancer Drug Des., 7:107-17 (1991), discloses processes for synthesizing a number of different PARP inhibitors. In addition, Suto et al., U.S. Pat. No. 5,177,075, discusses several isoquinolines used for enhancing the lethal effects of ionizing radiation or chemotherapeutic agents on tumor cells. Weltin et al., “Effect of 6(5H)-Phenanthridinone, an Inhibitor of Poly(ADP-ribose) Polymerase, on Cultured Tumor Cells”, Oncol. Res., 6:9, 399-403 (1994), discusses the inhibition of PARP activity, reduced proliferation of tumor cells, and a marked synergistic effect when tumor cells are co-treated with an alkylating drug. PARG inhibitors are known to be effective for treating cancer as described by the Japanese Patents of Tanuma. However, in direct contrast to the present invention, evidence in the literature suggest that the mechanism of action for treating cancer by PARG inhibitors is that PARG inhibitors prevent the PARG-associated degradation of PAR that normally blocks the transcription and activation of oncogenes.

Methods and compounds for inhibiting PARG are discussed in Tanuma et al., JP 042-75223-A2, “Poly(ADP-ribose)glycohydrolase Inhibitors Containing Glucose Derivatives”, Sep. 30, 1992; Tanuma et al., JP 042-75296-A2, “Adenosine Derivatives and their Use in Cancer Immunotherapy”, Mar. 4, 1991; Tanuma, JP 032-05402-A2, “Lignin Glycoside and Use”, Sep. 6, 1991; Tanuma, JP 04-013684-A2, “Lignin glycoside and Use”, Jan. 17, 1992; Slama et al., J. Med. Chem. 38: 389-393 (1995); Slama et al., J. Med. Chem. 38: 4332-4336 (1995); Maruta et al., Biochemistry 30:5907-5912 (1991); Aoki et al., Biochim. Biophys. Acta 1158:251-256 (1993); Aoki et al., Biochem. Biophys. Res. Comm. 210:329-337 (1995); Tsai et al., Biochemistry Intl. 24:889-897 (1991); and Concha et al., Biochemistry Intl. 24:889-897 (1991).

The use of the PARG inhibitor tannic acid for treating HIV infection is discussed in Uchiumi et al., “Inhibitory Effect of Tannic Acid on Human Immunodeficiency Virus Promoter Activity Induced by 12-O-Tetra Decanoylphorbol-13-acetate in Jurkat T-Cells”, Biochem. Biophys. Res. Comm. 220:411-417 (1996).

Still another use for PARP inhibitors is the treatment of peripheral nerve injuries, and the resultant pathological pain syndrome known as neuropathic pain, such as that induced by chronic constriction injury (CCI) of the common sciatic nerve and in which transsynaptic alteration of spinal cord dorsal horn characterized by hyperchromatosis of cytoplasm and nucleoplasm (so-called “dark” neurons) occurs. See Jianren Mao et al., 72:355-366 (1997). PARG inhibitors should influence PARP-associated neuropathic pain by downregulating PARP activity and thus PARG inhibitors are useful for treating or preventing peripheral nerve injuries, and the resultant pathological pain syndrome known as neuropathic pain and associated disorders or diseases as discussed herein.

PARP inhibitors have also been used to extend the lifespan and proliferative capacity of cells including treatment of diseases such as skin aging, Alzheimer's disease, atherosclerosis, osteoarthritis, osteoporosis, muscular dystrophy, degenerative diseases of skeletal muscle involving replicative senescence, age-related macular degeneration, immune senescence, AIDS, and other immune senescence diseases; and to alter gene expression of senescent cells. See WO 98/27975. PARG inhibitors should influence PARP-associated extension of the lifespan and proliferative capacity of cells by downregulating PARP activity and thus PARG inhibitors are useful for extending the lifespan and proliferative capacity of cells in a variety of circumstance including those diseases and disorders discussed herein.

Large numbers of known PARP inhibitors have been described in Banasik et al., “Specific Inhibitors of Poly(ADP-Ribose) Synthetase and Mono(ADP-Ribosyl)-Transferase”, J. Biol. Chem., 267:3, 1569-75 (1992), and in Banasik et al., “Inhibitors and Activators of ADP-Ribosylation Reactions”, Molec. Cell. Biochem., 138:185-97 (1994). Several PARG inhibitors have been described in Tavassoli et al., “Effect of DNA intercalators on poly(ADP-ribose) glycohydrolase activity”, Biochim Biophys. Acta 827:228-234 (1985).

However, the approach of using these PARG inhibitors to reduce NMDA-receptor stimulation, or to treat or prevent tissue damage resulting from cell damage or death due to necrosis or apoptosis, or to treat or prevent neural tissue damage caused by NO; ischemia and reperfusion of the heart or skeletal muscle; neural tissue damage resulting from ischemia and reperfusion injury; neurological disorders and neurodegenerative diseases; to prevent or treat vascular stroke; to treat or prevent cardiovascular disorders; to treat other conditions and/or disorders such as age-related macular degeneration, immune senescence diseases, arthritis, atherosclerosis, cachexia, degenerative diseases of skeletal muscle involving replicative senescence, diabetes, head trauma, immune senescence, inflammatory bowel disorders (such as colitis and Crohn's disease), muscular dystrophy, osteoarthritis, osteoporosis, pain (such as neuropathic pain), renal failure, retinal ischemia, septic shock (such as endotoxic shock), and skin aging; to extend the lifespan and proliferative capacity of cells; to alter gene expression of senescent cells; or to radiosensitize hypoxic tumor cells, has been limited in effect. For example, side effects have been observed with some of the best-known PARP inhibitors, as discussed in Milam et al., “Inhibitors of Poly(Adenosine Diphosphate-Ribose) Synthesis: Effect on Other Metabolic Processes”, Science, 223:589-91 (1984). Specifically, the PARP inhibitors 3-aminobenzamide and benzamide not only inhibited the action of PARP but also were shown to affect cell viability, glucose metabolism, and DNA synthesis. Thus, it was concluded that the usefulness of these PARP inhibitors may be severely restricted by the difficulty of finding a dose small enough to inhibit the enzyme without producing additional metabolic effects. Similar dose considerations may be also be concluded about PARG inhibitors.

Accordingly, there remains a need for compounds that inhibit PARG activity, compositions containing those compounds and methods utilizing those compounds, wherein the compounds produce more potent and reliable effects with fewer side effects, with respect to inhibiting PARG activity and treating the diseases and conditions discussed herein.

### SUMMARY OF THE INVENTION

The present invention is directed to a pharmaceutical composition comprising a PARG inhibitor or a pharmaceutically acceptable salt, hydrate, ester, solvate, prodrug, metabolite, or stereoisomer thereof, and a pharmaceutically acceptable carrier; wherein the PARG inhibitor is present in an amount that is effective for inhibiting or decreasing free radical induced cellular energy depletion, cell damage, or cell death and/or for the treatment or prevention of a disease or condition resulting from cell damage or death due to necrosis or apoptosis; and methods of using the same.

In a preferred embodiment, specific diseases and conditions suitable for treatment using the pharmaceutical compositions and methods of the present invention include acute pain, arthritis, atherosclerosis, cachexia, cardiovascular disorders, chronic pain, degenerative diseases, diabetes, diseases or disorders relating to lifespan or proliferative capacity of cells, diseases or disease conditions induced or exacerbated by cellular senescence, head trauma, immune senescence, inflammatory bowel disorders, ischemia, macular degeneration, muscular dystrophy, neural tissue damage resulting from ischemia and reperfusion injury, neurological disorders and neurodegenerative diseases, neuronal mediated tissue damage or disease, neuropathic pain, nervous insult, osteoarthritis, osteoporosis, peripheral nerve injury, renal failure, retinal ischemia, septic shock, skin aging, and vascular stroke.

In preferred embodiments of the invention, the PARG inhibitor may be glucose derivatives; lignin glycosides; hydrolysable tannins including gallotannins and ellagitannins; adenoside derivatives; acridine derivatives including 6,9-diamino-2-ethoxyacridine lactate monohydrate; tilorone analogs including tilorone R10.556, daunomycin or daunorubicin hydrochloride; ellipticine; proflavine; and other PARG inhibitors.

In a preferred embodiment, the PARG inhibitor is a glucose derivative, more particularly a compound of formula I:

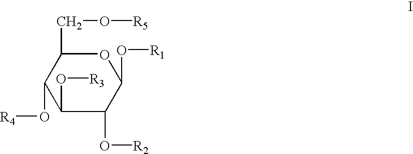

wherein:

R₁, R₂, R₃, R₄, R₅ individually represent a hydrogen atom or X,

X represents a carbonyl having a phenyl individually substituted by a plurality of groups selected from a group consisting of a hydroxyl group and C₁-C₈ alkoxy groups,

provided that R₁-R₅ do not represent a hydrogen atom simultaneously.

In still another preferred embodiment, the PARG inhibitor is a lignin glycoside, in particular a lignin glycoside having the following structure:

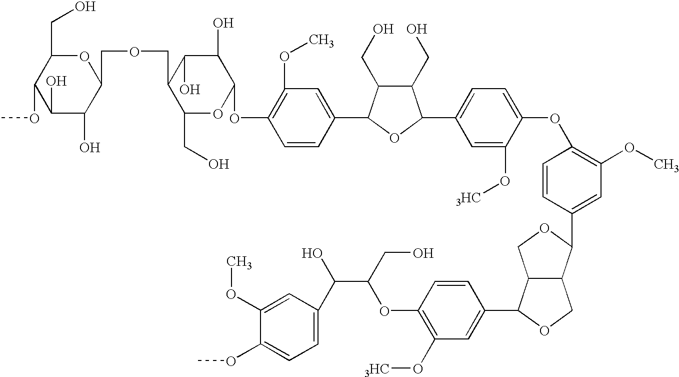

In another preferred embodiment, the PARG inhibitor is a hydrolysable tannin, particularly a hydrolysable tannin having the following properties:

(i) tannin and polysaccharide are bonded;

(ii) the molecular weight is 500 to 140,000;

(iii) the bonding ratio of tannin to polysaccharide is 1:1 to 20:1, as a molecular ratio;

(iv) the polysaccharide is composed of 60 to 70% uronic acid, and 30 to 40% neutral sugar.

Particularly preferred hydrolysable tannins include gallotannins and ellagitannins, especially those having the following properties:

(i) multiester formation of gallic acids and/or egallic acids and glucose; and

(ii) a molecular weight of approximately 700 to 8000.

In yet another preferred embodiment, the PARG inhibitor comprises an adenosine derivative, and more particularly an adenosine derivative. In a more preferred embodiment, the adenosine derivative is adenosine diphosphate-hydroxy-methyl-pyrrolidine-diol (also referred to as ADP-HPD) or a compound having the formula II:

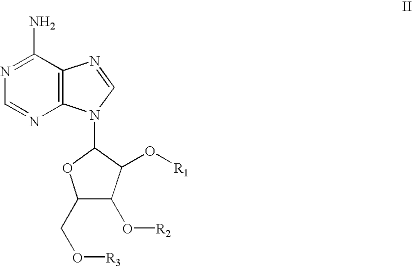

wherein:

R₁ represents a hydrogen atom, a group represented by formula III:

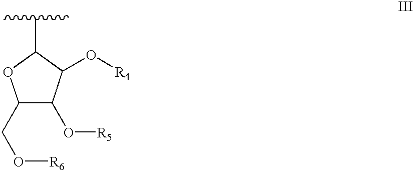

or X, wherein X is the compound of formula IV:

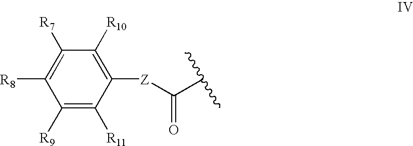

wherein Z is a bond, C₁-C₈ alkyl, or C₂-C₈ alkenyl; R₇, R₈, R₉, R₁₀, and R₁₁ are independently selected from hydrogen, hydroxyl, or C₁-C₈ alkoxy, provided that R₇-R₁₁ are not four or five hydrogen atoms simultaneously, and R₂, R₃, R₄, R₅, and R₆ independently represent a hydrogen atom or X, X representing the same as that described above; provided that R₁, R₂, and R₃ do not represent a hydrogen atom simultaneously; and further provided that R₂, R₃, R₄, R₅, and R₆ do not represent a hydrogen atom simultaneously.

In further preferred embodiments of the present invention, the PARG inhibitors may include acridine derivatives including 6,9-diamino-2-ethoxyacridine lactate monohydrate; tilorone analogs including tilorone R10.556, daunomycin or daunorubicin hydrochloride; ellipticine; proflavine; and other PARG inhibitors.

The invention further comprises methods of inhibiting or decreasing free radical induced cellular energy depletion, cell damage, or cell death and/or treating or preventing a disease or condition resulting from cell damage or death due to necrosis or apoptosis by administering an effective amount of a PARG inhibitor. In a preferred embodiment, specific diseases and conditions suitable for treatment using the pharmaceutical compositions and methods of the present invention include acute pain, arthritis, atherosclerosis, cachexia, cardiovascular disorders, chronic pain, degenerative diseases, diabetes, diseases or disorders relating to lifespan or proliferative capacity of cells, diseases or disease conditions induced or exacerbated by cellular senescence, head trauma, immune senescence, inflammatory bowel disorders, ischemia, macular degeneration, muscular dystrophy, neural tissue damage resulting from ischemia and reperfusion injury, neurological disorders and neurodegenerative diseases, neuronal mediated tissue damage or disease, neuropathic pain, nervous insult, osteoarthritis, osteoporosis, peripheral nerve injury, renal failure, retinal ischemia, septic shock, skin aging, and vascular stroke.

In a particularly preferred embodiment, the compositions described above as PARG inhibitors are used in the methods of the present invention.

### BRIEF DESCRIPTION OF THE FIGURES

FIG. 1 is a graph showing protective effect of the pharmaceutical compositions of the present invention against hydrogen peroxide cytotoxicity.

FIG. 2 shows the EC₅₀ as determined from a cytotoxicity dose responsive curve.

FIG. 3 is a schematic simplified representation of the PARP/PARG cycle for maintenance of poly(ADP-ribosyl)ation and its relationship to cellular energy metabolism and the various uses, diseases and disorders described herein.

### DETAILED DESCRIPTION OF THE INVENTION

It has been unexpectedly discovered that PARG inhibitors can be used to inhibit or decrease free radical induced cellular energy depletion, cell damage, or cell death and/or treat or prevent a disease or condition resulting from cell damage or death due to necrosis or apoptosis. In particular, PARG inhibitors can be administered in effective amounts to treat or prevent specific diseases and conditions including acute pain, arthritis, atherosclerosis, cachexia, cardiovascular disorders, chronic pain, degenerative diseases, diabetes, diseases or disorders relating to lifespan or proliferative capacity of cells, diseases or disease conditions induced or exacerbated by cellular senescence, head trauma, immune senescence, inflammatory bowel disorders, ischemia, macular degeneration, muscular dystrophy, neural tissue damage resulting from ischemia and reperfusion injury, neurological disorders and neurodegenerative diseases, neuronal mediated tissue damage or disease, neuropathic pain, nervous insult, osteoarthritis, osteoporosis, peripheral nerve injury, renal failure, retinal ischemia, septic shock, skin aging, and vascular stroke.

For example, we have discovered that PARG inhibitors can be used to treat or prevent cardiovascular tissue damage resulting from cardiac ischemia or reperfusion injury. Reperfusion injury, for instance, occurs at the termination of cardiac bypass procedures or during cardiac arrest when the heart, once prevented from receiving blood, begins to reperfuse.

The PARG inhibitors of the present invention can also be used to extend or increase the lifespan or proliferation of cells and thus to treat or prevent diseases associated therewith and induced or exacerbated by cellular senescence including skin aging, atherosclerosis, osteoarthritis, osteoporosis, muscular dystrophy, degenerative diseases of skeletal muscle involving replicative senescence, age-related macular degeneration, immune senescence, and other diseases associated with cellular senescence and aging, as well as to alter the gene expression of senescent cells.

Preferably, the PARG inhibitors are used in the present invention to treat or prevent tissue damage resulting from cell death or damage due to necrosis or apoptosis; to treat or prevent neural tissue damage resulting from cerebral ischemia and reperfusion injury or neurodegenerative diseases in a mammal; to extend and increase the lifespan and proliferative capacity of cells; and to alter gene expression of senescent cells.

Calcium overload and poly (ADP-ribose) polymerase activation plays a role in the disruption of energy homeostasis leading to cell death, elevated intracellular calcium (Ca²⁺) elicits cytotoxicity through downstream generation of reactive nitrogen and oxygen species which disrupt energy homeostasis through several modes of cellular damage. Ca²⁺ can enter the cytoplasm through voltage- or ligand-gated ion channels, such as the NMDA-subtype glutamate receptor. ATP is required for the removal of calcium from the cytoplasm via ion-motive ATPases which either pump Ca²⁺ out of the cell or into endoplasmic reticulum (ER). Mitochondria also help buffer cytoplasmic calcium. Excessive accumulation of Ca²⁺ by mitochondria impairs oxidative phosphorylation, while also promoting production of reactive oxygen species, such as superoxide (O²⁻) and hydrogen peroxide (H₂O₂), via the electron transport chain. High mitochondrial Ca²⁺ accumulation also alters permeability of the mitochondrial membrane, which inhibits mitochondrial ATP production and promotes necrosis. In addition, selective permeability of the outer membrane releases cytochrome C (Cyt C) which activates caspases¹⁰³. Caspases, in turn, cleave specific cytoplasmic and nuclear protein substrates to coordinate apoptosis (see text). Ca²⁺ also directly activates several cellular enzymes that initiate cytotoxic cascades. These include the Ca²⁺/Mg²⁺ activated endonuclease (DNase) as well as Ca²⁺ sensitive phospholipases and proteases. In addition several Ca²⁺ activated enzymes are involved in free radical production. Ca²⁺ activated proteases known as calpains convert xanthine dehydrogenase to xanthine oxidase (XO) which promotes enzymatic generation of superoxide. Cyclooxygenases are another source of superoxide. Hydrogen peroxide (H₂O₂) can be formed from superoxide and can itself be converted to the highly reactive hydroxyl radical (OH) via iron catalyzed reactions. These reactive oxygen species damage lipids, proteins and nucleic acids. Ca²⁺ also activates the calmodulin-regulated enzyme nitric oxide synthase (NOS) to produce large amounts of nitric oxide (NO). Superoxide and nitric oxide combine to form the much more reactive peroxynitrite anion (OONO—). Peroxynitrite damages the cell membrane and leads to oxidation and nitration of proteins containing aromatic amino acids such as tyrosine. Peroxynitrite also provides another route for the formation of hydroxyl radicals, most likely through a peroxynitrous acid intermediate. DNA damage produced by either the Ca²⁺/Mg²⁺ activated endonuclease, OONO—, or by hydroxyl radicals results in robust PARP activation with subsequent depletion of NAD levels. Since NAD is required for ATP production and since ATP is, in turn, required for NAD synthesis, excessive PARP activation depletes the cellular energy pool and results in cell death.

Evidence in the literature suggests that PARG inhibitors inhibit PARG by directly interacting with the PARG enzyme, the PAR polymer, or both. PARG inhibitors may also be useful for the methods described herein by a mechanism of action independent of a direct interaction between the inhibitor and PARG.

In a preferred embodiment of the present invention, the poly(ADP-ribose)glycohydrolase inhibitors contain as active ingredients glucose derivatives; lignin glycosides; hydrolysable tannins including gallotannins and ellagitannins; adenoside derivatives; acridine derivatives including 6,9-diamino-2-ethoxyacridine lactate monohydrate; tilorone analogs including tilorone R10.556, daunomycin or daunorubicin hydrochloride; ellipticine; proflavine; and other PARG inhibitors. Other preferred embodiments of the present invention are directed to the use of PARG inhibitors, particularly those described herein and others well known in the art, and their method of use in treating or preventing diseases or conditions due to free radical induced cellular energy depletion and/or tissue damage resulting from cell damage or death due to necrosis, apoptosis, or combinations thereof.

Particularly preferred PARG inhibitors include glucose derivatives, especially those glucose derivatives of the type represented by the general formula (I):

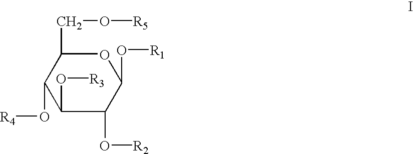

wherein R₁-R₅ individually represent a hydrogen atom or X, X representing a carbonyl having a phenyl substituted by a plurality of groups selected from a group consisting of a hydroxyl group and lower-alkoxy groups, provided that R₁-R₅ do not represent a hydrogen atom simultaneously.

In another preferred embodiment of the present invention, lower alkoxy represented by X preferably contain from one to four carbons and specifically include methoxy, ethoxy, propoxy, iso-propoxy, butoxy, iso-butoxy, sec-butoxy, tertbutoxy, and the like. In particular, methoxy is preferred.

As X, those in which a phenyl is bound to a carbonyl via alkylene or alkenylene and those in which a phenyl is directly bound to a carbonyl are particularly preferred. As to alkylenes, those containing one to four carbons, such as methylene, ethylene, trimethylene, and tetramethylene, are exemplified, and methylene and ethylene are particularly preferable. As to alkenylenes, those containing one to four carbons are exemplified and vinylene is particularly preferred.

Preferred examples of X are groups represented by the following general formula:

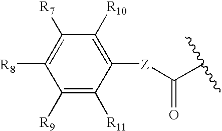

wherein Z represents a direct bond, alkylene, or alkenylene, R₇-R₁₁ individually represent a hydrogen atom, a hydroxyl group or a lower alkoxy, provided that R₇-R₁₁ do not represent 4 or 5 hydrogen atoms simultaneously.

Specific examples of X which are particularly preferable are galloyl, 4-hydroxy-3-methoxybenzoyl, 4-hydroxy-3,5-dimethoxybenzoyl, 3,4,5-trimethoxybenzoyl, 4-hydroxy-3-methoxycinnamoyl, 4-hydroxy-3,5-dimethoxycinnamoyl, 3,4,5-trimethoxycinnamoyl, 3,4,5-trihydroxybenzylcarbonyl, and 3, 4,5-trihydroxyphenetylcarbonyl.

A preferred emodiment of the glucose derivatives is 1,2,3,4,6-Penta-O-Galloyl-Glucose and has the following structure:

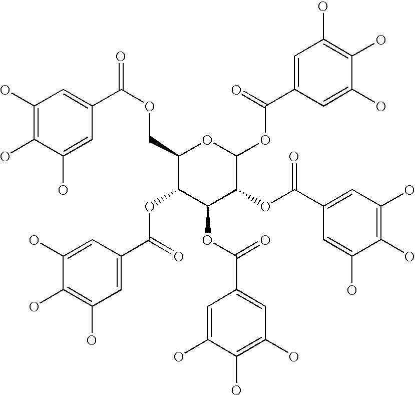

These glucose derivatives useful as PARG inhibitors in the present invention are can be prepared in any suitable manner known to one of ordinary skill in the art from readily available materials. In particular, they can be prepared in the following manner:

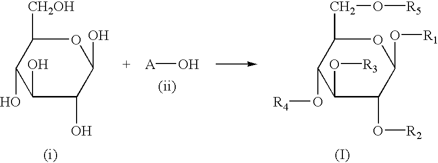

wherein X is the same as those described above. The above reaction takes place by an ordinary ester reaction.

Compound (i) and compound (ii) as starting materials are both well known in the art and are readily available. The above compound (i) is glucose, and the compound (ii) is a carboxylic acid.

Hydrolysable tannins and lignin glycosides suitable for use in the invention may be prepared in any manner known in the art and may be prepared in the following manner.

As a starting material, any suitable organic matter, such as, pinecones, tea leaves, grass dogwood, trisaccharide root, and the like, can be treated in a suitable solvent, such as hot water, ethanol, acetone for about 1 to 15 hours. The treated material is extracted in an alkaline solution (0.1 to 1N sodium hydroxide, ammonium, and the like). The extracted liquid is adjusted to pH 4 to 6, and an equivalent amount of ethanol is added, and the supernatant fraction is recovered. The supernatant fraction is refined by gel filtration, and the active portion is recovered. The hydrolysable tannin or lignin glycoside obtained can then be treated by dialysis, centrifugal separation, freeze-drying, etc.

Suitable hydrolysable tannins and lignin glycosides have poly-(ADPribose) glycohydrolase inhibitory action, and presents poly-(ADP-ribose) glycohydrolase inhibitory activity to mammals and is useful for inhibiting or decreasing free radical induced cellular energy depletion, cell damage or cell death. Hydrolysable tannins and lignin glycosides useful in the pharmaceutical compositions and methods of the invention may be administered either orally or parenterally, preferably with a suitable carrier in the form of a pharmaceutical composition. Such hydrolysable tannins and lignin glycosides may be administered, for example, by oral route, usually by about 0.1 to 100 mg/kg of body weight a day either once or in several divided portions, but the dose maybe varied depending on the age, body weighs and/or severity of the disease to be treated and reaction to treatment.

The toxicity of these hydrolyzable tannin glycosides has been investigated, and, by oral administration, the LD50 value was 100 mg/kg or more, which is extremely high resulting in a broad safety region.

Suitable acridine derivatives include compounds having the formula have the following structure:

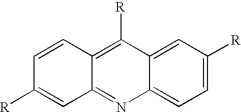

wherein R is independently selected from hydrogen, halo, alkylhalo, hydroxy, C₁-C₆ straight or branched chain alkyl, C₂-C₆ straight or branched chain alkenyl group, C₁-C₆ straight or branched chain alkoxy, C₂-C₆ straight or branched chain alkenoxy group, amino, C₁-C₆ alkylamino, C₁-C₆ alkylthio, thio, nitro, nitroso, carboxy; wherein said alkyl, alkenyl, alkoxy, alkenoxy, alkylamino, alkylhalo and alkylthio groups are independently substituted with one or more substituent(s) selected from halo, hydroxy, amino, thio, nitro, C₁-C₄ alkoxy, or C₂-C₄ alkenyloxy.

Other suitable PARG inhibitors include adenoside derivatives; acridine derivatives including 6,9-diamino-2-ethoxyacridine lactate monohydrate; tilorone analogs including tilorone R10.556, daunomycin or daunorubicin hydrochloride; ellipticine; proflavine; and other PARG inhibitors known in the art.

PARG inhibitors, particularly as described above, possess a poly(ADP-ribose)glycohydrolase activity as shown by the experimental examples given below and are especially useful as poly(ADP-ribose)glycohydrolase inhibitors for inhibiting or decreasing free radical induced cellular energy depletion, cell damage, or cell death and/or treating or preventing a disease or condition resulting from cell damage or death due to necrosis or apoptosis. In particular, PARG inhibitors can be administered in effective amounts to treat or prevent specific diseases and conditions including acute pain, arthritis, atherosclerosis, cachexia, cardiovascular disorders, chronic pain, degenerative diseases, diabetes, diseases or disorders relating to lifespan or proliferative capacity of cells, diseases or disease conditions induced or exacerbated by cellular senescence, head trauma, immune senescence, inflammatory bowel disorders, ischemia, macular degeneration, muscular dystrophy, neural tissue damage resulting from ischemia and reperfusion injury, neurological disorders and neurodegenerative diseases, neuronal mediated tissue damage or disease, neuropathic pain, nervous insult, osteoarthritis, osteoporosis, peripheral nerve injury, renal failure, retinal ischemia, septic shock, skin aging, and vascular stroke.

The PARG inhibitors suitable for use in the present invention include glucose derivatives; lignin glycosides; hydrolysable tannins including gallotannins and ellagitannins; adenoside derivatives; acridine derivatives including 6,9-diamino-2-ethoxyacridine lactate monohydrate; tilorone analogs including tilorone R10.556, daunomycin or daunorubicin hydrochloride; ellipticine; proflavine; and other PARG inhibitors. The invention includes pharmaceutical compositions containing PARG inhibitors and their method of use in treating or preventing diseases or conditions due to free radical induced cellular energy depletion and/or tissue damage resulting from cell damage or death due to necrosis, apoptosis, or combinations thereof.

The PARG inhibitors suitable for use in the present invention may be useful in a free base form, in the form of pharmaceutically acceptable salts, pharmaceutically acceptable hydrates, pharmaceutically acceptable esters, pharmaceutically acceptable solvates, pharmaceutically acceptable prodrugs, pharmaceutically acceptable metabolites, and in the form of pharmaceutically acceptable stereoisomers. These forms are all within the scope of the invention. In practice, the use of these forms amounts to use of the neutral compound.

“Pharmaceutically acceptable salt”, “hydrate”, “ester” or “solvate” refers to a salt, hydrate, ester, or solvate of the inventive PARG inhibitors which possesses the desired pharmacological activity and which is neither biologically nor otherwise undesirable. Organic acids can be used to produce salts such as acetate, adipate, alginate, aspartate, benzoate, benzenesulfonate, p-toluenesulfonate, bisulfate, sulfamate, sulfate, naphthylate, butyrate, citrate, camphorate, camphorsulfonate, cyclopentane-propionate, digluconate, dodecylsulfate, ethanesulfonate, fumarate, glucoheptanoate, glycerophosphate, hemisulfate heptanoate, hexanoate, 2-hydroxyethanesulfonate, lactate, maleate, methanesulfonate, 2-naphthalenesulfonate, nicotinate, oxalate, tosylate and undecanoate. Inorganic acids can be used to produce salts such as hydrochloride, hydrobromide, hydroiodide, and thiocyanate.

Examples of suitable base salts include hydroxides, carbonates, and bicarbonates of ammonia, alkali metal salts such as sodium, lithium and potassium salts, alkaline earth metal salts such as calcium and magnesium salts, aluminum salts, and zinc salts.

Salts may also be formed with organic bases. Organic bases suitable for the formation of pharmaceutically acceptable base addition salts of the PARG inhibitors of the present invention include those that are non-toxic and strong enough to form such salts. For purposes of illustration, the class of such organic bases may include mono-, di-, and trialkylamines, such as methylamine, dimethylamine, triethylamine and dicyclohexylamine; mono-, di- or trihydroxyalkylamines, such as mono-, di-, and triethanolamine; amino acids, such as arginine and lysine; guanidine; N-methyl-glucosamine; N-methyl-glucamine; L-glutamine; N-methyl-piperazine; morpholine; ethylenediamine; N-benzyl-phenethylamine; (trihydroxy-methyl)aminoethane; and the like. See, for example, “Pharmaceutical Salts,” J. Pharm. Sci., 66:1, 1-19 (1977). Accordingly, basic nitrogen-containing groups can be quaternized with agents including: lower alkyl halides such as methyl, ethyl, propyl, and butyl chlorides, bromides and iodides; dialkyl sulfates such as dimethyl, diethyl, dibutyl and diamyl sulfates; long chain halides such as decyl, lauryl, myristyl and stearyl chlorides, bromides and iodides; and aralkyl halides such as benzyl and phenethyl bromides.

The acid addition salts of the basic PARG inhibitors may be prepared either by dissolving the free base of a PARG inhibitor in an aqueous or an aqueous alcohol solution or other suitable solvent containing the appropriate acid or base, and isolating the salt by evaporating the solution. Alternatively, the free base of the PARG inhibitor may be reacted with an acid, as well as reacting the PARG inhibitor having an acid group thereon with a base, such that the reactions are in an organic solvent, in which case the salt separates directly or can be obtained by concentrating the solution.

“Pharmaceutically acceptable prodrug” refers to a derivative of the inventive PARG inhibitors which undergoes biotransformation prior to exhibiting its pharmacological effect(s). The prodrug is formulated with the objective(s) of improved chemical stability, improved patient acceptance and compliance, improved bioavailability, prolonged duration of action, improved organ selectivity, improved formulation (e.g., increased hydrosolubility), and/or decreased side effects (e.g., toxicity). The prodrug can be readily prepared from the inventive PARG inhibitors using methods known in the art, such as those described by Burger's Medicinal Chemistry and Drug Chemistry, Fifth Ed., Vol. 1, pp. 172-178, 949-982 (1995). For example, the inventive PARG inhibitors can be transformed into prodrugs by converting one or more of the hydroxy or carboxy groups into esters.

After entry into the body, most drugs are substrates for chemical reactions that may change their physical properties and biologic effects. These metabolic conversions, which usually affect the polarity of the PARG inhibitor, alter the way in which drugs are distributed in and excreted from the body. However, in some cases, metabolism of a drug is required for therapeutic effect. For example, anticancer drugs of the antimetabolite class must be converted to their active forms after they have been transported into a cancer cell.

Since must drugs undergo metabolic transformation of some kind, the biochemical reactions that play a role in drug metabolism may be numerous and diverse. The main site of drug metabolism is the liver, although other tissues may also participate.

A feature characteristic of many of these transformations is that the metabolic products, or “metabolites”, are more polar than the parent drugs, although a polar drug does sometimes yield a less polar product. Substances with high lipid/water partition coefficients, which pass easily across membranes, also diffuse back readily from tubular urine through the renal tubular cells into the plasma. Thus, such substances tend to have a low renal clearance and a long persistence in the body. If a drug is metabolized to a more polar compound, one with a lower partition coefficient, its tubular reabsorption will be greatly reduced. Moreover, the specific secretory mechanisms for anions and cations in the proximal renal tubules and in the parenchymal liver cells operate upon highly polar substances.

As a specific example, phenacetin (acetophenetidin) and acetanilide are both mild analgesic and antipyretic agents, but are transformed within the body to a more polar and more effective metabolite, p-hydroxyacetanilid (acetaminophen), which is widely used today. When a dose of acetanilid is given to a person, the successive metabolites peak and decay in the plasma sequentially. During the first hour, acetanilid is the principal plasma component. In the second hour, as the acetanilid level falls, the metabolite acetaminophen concentration reaches a peak. Finally, after a few hours, the principal plasma component is a further metabolite that is inert and can be excreted from the body. Thus, the plasma concentrations of one or more metabolites, as well as the drug itself, can be pharmacologically important.

The reactions involved in drug metabolism are often classified into two groups, as shown in the Table II. Phase I reactions are functionalization reactions and generally consist of (1) oxidative and reductive reactions that alter and create new functional groups and (2) hydrolytic reactions that cleave esters and amides to release masked functional groups. These changes are usually in the direction of increased polarity.

Phase II reactions are conjugation reactions in which the drug, or often a metabolite of the drug, is coupled to an endogenous substrate, such as glucuronic acid, acetic acid, or sulfuric acid.

TABLE II
Phase I Reactions (functionalization reactions):

(1) Oxidation via the hepatic microsomal P450 system:

Aliphatic oxidation
Aromatic hydroxylation
N-Dealkylation
O-Dealkylation
S-Dealkylation
Epoxidation
Oxidative deamination
Sulfoxide formation
Desulfuration
N-Oxidation and N-hydroxylation
Dehalogenation

(2) Oxidation via nonmicrosomal mechanisms:

Alcohol and aldehyde oxidation
Purine oxidation
Oxidative deamination (monoamine oxidase and
diamine oxidase)

(3) Reduction:

Azo and nitro reduction

(4) Hydrolysis:

Ester and amide hydrolysis
Peptide bond hydrolysis
Epoxide hydration

Phase II Reactions (conjugation reactions):

(1) Glucuronidation
(2) Acetylation
(3) Mercapturic acid formation
(4) Sulfate conjugation
(5) N-, O-, and S-methylation
(6) Trans-sulfuration

Where a PARG inhibitor possesses one or more asymmetric center(s) and thus can be produced as mixtures (racemic and non-racemic) of stereoisomers, or as individual R- and S-stereoisomers. The individual stereoisomers may be obtained by using an optically active starting material, by resolving a racemic or non-racemic mixture of an intermediate at some appropriate stage of synthesis, or by resolving a desired PARG inhibitor compound. The term “isomers” refer to compounds having the same number and kind of atoms, and hence, the same molecular weight, but differing in respect to the arrangement or configuration of the atoms. “Stereoisomers” are isomers that differ only in the arrangement of atoms in space. “Enantiomers” are a pair of stereoisomers that are non-superimposable mirror images of each other.

“Diastereoisomers” are stereoisomers which are not mirror images of each other. “Racemic mixture” means a mixture containing equal, or roughly equal, parts of individual enantiomers. A “non-racemic mixture” is a mixture containing unequal, or substantially unequal, parts of individual enantiomers or stereoisomers.

Synthesis of Compounds

PARG inhibitors suitable for use in the pharmaceutical compositions and methods of the present invention may be synthesized by known methods from starting materials that are known, are themselves commercially available, or may be prepared by methods used to prepare corresponding compounds in the literature.

The compounds of the present invention can also be readily prepared by standard techniques of organic chemistry, using the general synthetic pathways depicted below. Precursor compounds can be prepared by methods known in the art. The following schemes are intended as illustrations of the preparation of suitable PARG inhibitors useful in preferred embodiments of the invention, and no limitation of the invention is implied.

### EXAMPLE 1

1. Synthesis of 1-O-Benzyl-D-Glucopyranose (Compound 1)

D-glucose (15.0 g) was added to benzyl alcohol (100 ml) and the suspension thus obtained was cooled to 0° C. Hydrogen chloride gas was then blown into the suspension for 30 minutes. After the resulting solution was stirred at room temperature for 2 days, ether (500 ml) was added and the supernatant liquid was decanted. This process was repeated 3 times. The oily substance thus obtained was subjected to silica gel column chromatography (silica gel, solvent: chloroform:methanol=8/1, 5/1) to obtain Compound 1 (11.7 g, 52%).

2. Synthesis of 3, 4,5-Tribenzyloxybenzoic Acid (Compound 2)

A solution obtained by mixing dimethylformamide (50 ml), gallic acid (10 g), anhydrous potassium carbonate (44 g), and benzyl chloride (27 ml) under nitrogen atmosphere was diluted with ethyl acetate (1 liter). Then, the mixture was stirred at 140° C. overnight. The ethyl acetate layer was washed with water and a saturated saline solution, and dried with magnesium sulfate. After the solvent was distilled off under reduced pressure, a crude product was obtained. Ethanol (200 ml) and a 1.6N sodium hydroxide water solution (50 ml) were added to the crude product thus obtained, and the mixture was reflexed under heating for 2 hours. After reaction, about 50% of ethanol was distilled off. The resulting sediment was cooled to 0° C. and adjusted to pH 2 with 0.5 N hydrochloric acid. The solids thus deposited were filtered off and dried to obtain Compound 2 (15.6 g, 64%).

3. Synthesis of 1-O-Benzyl-2,3,4,6-Tetrakis-(3,4,5-Tribenzyloxybenzoyl)-D-Glucopyranose (Compound 3)

Compound 2 (7.0 g), thionyl chloride (40 ml), and dimethylformamide (1 ml) were mixed under ice cooling. After the resultant solution was refluxed under heating overnight, excessive thionyl chloride was distilled off under ordinary pressure and reduced pressure to prepare an acid chloride of Compound 2. Under nitrogen atmosphere, Compound 1 (0.83 g) was added to pyridine (10 ml) and the mixture was stirred. To the solution, a solution of the acid chloride of Compound 2 (a crude product obtained when 7.0 g of Compound 2 was employed) in pyridine (30 ml) was dropped. The mixture was stirred at room temperature overnight and diluted with ethyl acetate (0.6 liter). The suspension thus obtained was filtered. The ethyl acetate layer was washed with water, 0.05 N hydrochloric acid, a saturated sodium hydrogen carbonate water solution, and a saturated saline solution, and then, dried with magnesium sulfate. After the solvents were distilled off under reduced pressure, a crude product was obtained. The crude product thus obtained was subjected to silica gel column chromatography (silica gel, solvents: ethyl acetate:hexane=1/4, 1/3, 1/2) to obtain Compound 3 (2.85 g, 49%).

¹H-NMR (CDCl₃)δ: 4.2-4.8 (m, 3H), 4.8-5.1 (m, 24H), 5.1-5.7 (m, 3H), 6.1-6.3 (m, 1H), and 7.1-7.6 (m, 72H).

IR (KBr, cm⁻¹): 1,718 and 1,580

4. Synthesis of 2,3,4,6-Tetrakis-O-Galloyl-D-Glucopyranose (Compound 4)

After mixing Compound 3 (2.85 g), ethyl acetate/methanol (3/1, 150 ml), and palladium-black (3.0 g), hydrogen substitution was initiated. After the reaction mixture was stirred at room temperature for about 1 hour, palladium-black was removed. The resulting filtrate was concentrated and subjected to silica gel column chromatography (silica gel, solvent: hexane:tetrahydrofuran:methanol=60/30/10, 50/37.5/12.5, 40/45/15) to obtain Compound 4 (0.94 g, 86%).

¹H-NMR (DMSO-d6)δ: 4.3-4.5 (m, 2H), 5.0-5.2 (m, 2H), 5.3-5.5 (m, 2H), 5.8-6.2 (m, 1H, H¹), 6.7-7.1 (m, 8H), and 9.19 (brs, 12H)

IR (KBr, cm⁻¹): 3,300, 1,700, and 1,610

¹³C-NMR (DMSO-d6)δ: 62.0, 66.2, 67.0, 68.4, 69.4, 89.5, 104.2, 108.8, 116.2, 116.3, 116.5, 116.6, 119.1, 138.6, 138.7, 139.0, 142.8, 143.0, 145.3, 145.5, 145.6, 164.5, 164.7, 165.0, 165.2, and 165.5 (mixture of α and β).

### EXAMPLE 2

Synthesis of 1,2,3,4,5-Penta-O-Galloyl-β-D-Glucopyranose (Compound 5)

Tannic acid (25 g), methanol (200 ml) and 0.1 M acetic acid-sodium acetate (pH 6.0, 200 ml) were mixed and reaction was allowed to proceed in a thermostat at 37° C. for 7 days with occasional stirring. After reaction, the solution was concentrated to reduce the volume to about 50% and the resulting concentrated solution was extracted with ethyl acetate. The resultant extract was washed with water and a saturated saline solution, and then, dried with magnesium sulfate. After the solvent was distilled off, a crude product (about 20 g) was obtained. The resulting crude product (10 g) was subjected to silica gel column chromatography (silica gel, solvents: hexane:tetrahydrofuran:methanol=6/3/1, 50/37.5/12.5, 4/4.5/1.5) to obtain Compound 5 (1.39 g).

¹H-NMR (DMSO-d₆)δ: 4.3 (brs), 4.5-4.6 (m), 5.94 (d, d, J=9.7), 6.35 (d, J=8.3 Hz, 1H), 6.77 (s, 2H), 6.82 (s, 2H), 6.85 (s, 2H), 6.92 (s, 2H), 6.98 (s, 2H), find 9.11 (brs, 15H).

IR (KBr, cm⁻¹): 3,350, 1,700, and 1,610

¹³C-NMR (DMSO-d₆)δ: 61.3, 67.6, 70.5, 71.9, 72.2, 91.7, 108.8, 117.4, 118.0, 118.1, 118.9, 138.6, 138.8, 139.0, 139.5, 145.3, 145.3, 145.4, 145.6, 163.9, 164.4, 164.6, 164.8, and 165.4.

### EXAMPLES 3 TO 5

The following three compounds were synthesized according to Example 1.

### EXAMPLE 3

1,2,3,4,6-Penta-O-(3,5-Dimethoxy-4-Hydroxycinnamoyl)-D-Gluropyranose

¹H-NMR (CDCl₃/D₂O)δ: 3.78-4.01 (m, 30H), 4.27-6.90 (m, 7H), 6.13-6.55 (m, 5H), 6.60-6.90 (m, 10H), and 7.46-7.80 (m, 5H).

IR (KBr, cm⁻¹): 2,950, 2,850, 1,710, 1,630, 1,600, 1,510, 1,460, 1,280, and 1,220.

### EXAMPLE 4

1,2,3,4,6-Penta-O-(3,4,5-Trimethoxybenzoyl)-D-Glucopyranose

¹H-NMR (CDCl₃)δ: 3.8-4.1 (m, 45H), 4.3-4.9 (m, 3H), 5.57 (dd, 0.4H), 5.7-5.9 (m, 1.6H), 5.9-6.2 (m, 0.6H), 6.2-6.4 (m, 1H), 6.81 (d, 0.4H), and 7.1-7.5 (m, 10H).

IR (KBr, cm⁻¹): 1,720, 1,580, 1,330, 1,210, and 1,125 mp. 85-90° C.

### EXAMPLE 5

1,2, 3, 4,6-Penta-O-(3,4,5-Trimethoxycinnamoyl)-D-Glucose

¹H-NMR (CDCl₃)δ: 3.60-4.05 (m, 45H), 4.30-6.97 (m, 7H), 6.19-6.55 (m, 5H), and 6.60-6.97 (10H).

IR (KBr, cm⁻¹): 2,930, 1,720, 1,630, 1,580, 1,500, 1,270, 1,240, and 1,130.

### EXAMPLE 6

Preparation of Hydrolysable Tannins from Pinecones

Pinecones are extracted in hot water by boiling with the boiling time varying with the amount of pinecones and/or the amount of water, but is usually 2 hours×3 times. After the pinecones are extracted in hot water, they are half dried, and immersed in ethanol, and allowed to stand overnight at room temperature. After extraction of the pinecones in ethanol, the pinecones are half dried, and the resultant hydrolysable tannin or lignin glycoside is extracted by immersion in acetone, and allowed to stand overnight at room temperature, dried by lamp, and extracted in 1N sodium hydroxide solution while stirring for 6 hours (or overnight). Acetic acid is added to this extracted solution, and the pH is returned to 5.0. The precipitate is removed by high speed centrifugal operation. An equivalent amount of ethanol is added to the extracted solution, and let stand overnight in a cold room. The precipitate is removed by high speed centrifugal operation, and the supernatant is dialyzed in water. The dialyzed solution is freeze-dried, and powder is obtained. The freeze-dried powder is refined by Sepharose CL-4B (the moving bed is 0.1 N NaOH). Active fractions are collected and dialyzed in water, and freeze-dried, and powder is obtained. This freeze-cried powder is dissolved in 10% ethanol, and is further refined by Toyopearl HW-40F (the moving bed is 10% ethanol). Active fractions are collected, dialyzed in wafer, and freeze-dried, end powder is obtained.

### TEST EXAMPLE 1

Inhibitory Effect on poly-(ADP-ribose) Glycohydrolase

To a buffer for assay (0.01 bovine serum albumin, 10 mM mercaptoethanol, 50 mM potassium phosphate, pH 7.0), 3H-(ADP-ribose) n=15 was added, and to 27 pl thereof, further, the substance to be tested and nuclear derivative poly-(ADP-ribose) glycohydrolase solution prepared from human placenta were added to make up 30 pl in total, which was incubated for 1 hour at 37° C. Later, the reaction solution was absorbed in DE81 filter paper, and the filter paper was washed in water, ethanol and acetone, and was dried, and the unreacted substrate 3H(ADP-ribose) was measured by liquid scintillation counter, and the inhibitory action of the test substance on this enzyme was investigated. Results are shown in Table 1, which shows all tested substances inhibited poly-(ADP-ribose) glycohydrolase dose-dependently.

TABLE 1
Inhibitory activity of tannin glycoside on
poly-(ADP-ribose) glycohydrolase

| Concentration of tannin | | Activity of PARG (%) |
|---|---|---|
| | 0 | 100 |
| | 0.3 | 86 |
| | 1.0 | 24 |
| | 3.0 | 4 |

Other variations and modifications of this invention using, among others, the synthetic pathways described above will be obvious to those skilled in the art.

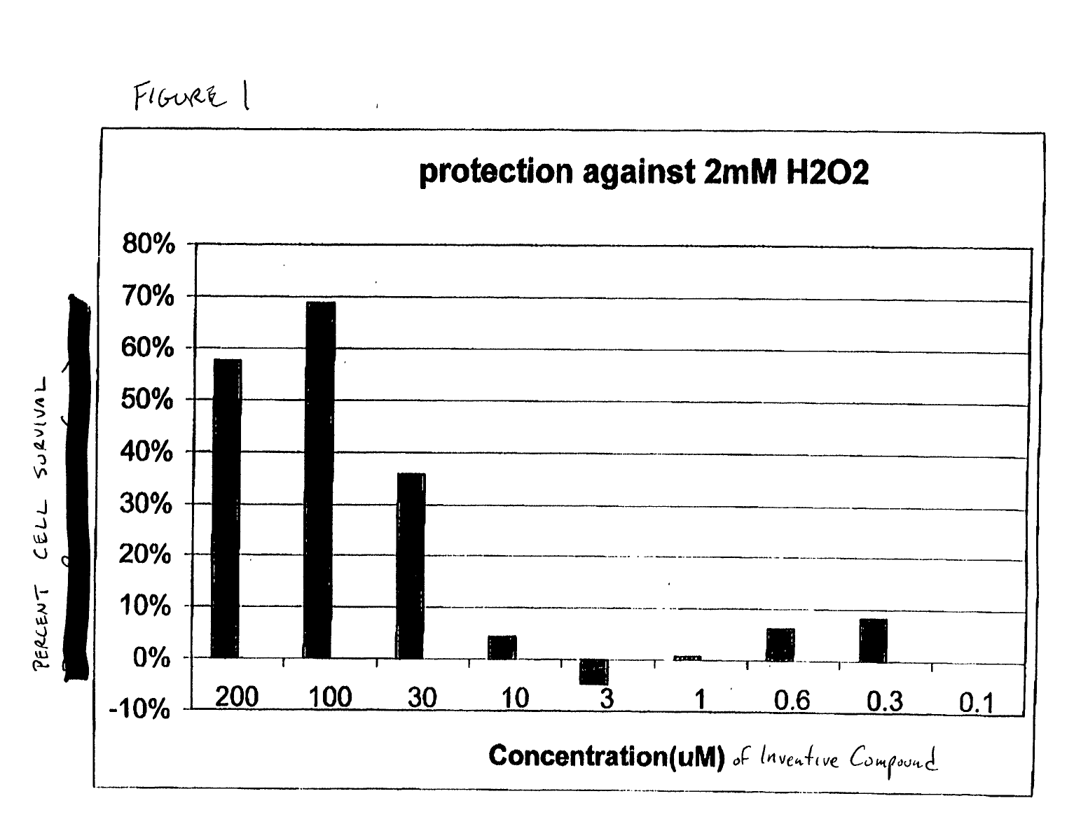

FIG. 1 shows P388D1 cells (ATCC, #CCL-46), derived from murine macrophage like tumor, were maintained in Dulbeco's Modified Eagle Medium (DMEM) with 10% horse serum, 2 mM L-glutamine. The cytotoxicity assay was set up in a 96-well plate. In each well, 190 ul cells were seeded at 2×10⁶/ml density. A dose responsive experiment was conducted. Various concentration of a PARG inhibitor was added to the cells. A typical experiment consisted of doses with a final concentrations of 0.01, 0.03, 0.1, 0.3, 1, 3, 10, 30, 100, 200 uM. Each data point was averaged from a quadruplicate. After 15 min incubation, 5 ul of freshly prepared hydrogen peroxide were added to the cells to a final concentration of 2 mM. A set of wells with no compound was not exposed to hydrogen peroxide for background determination. Cells were returned to 37° C. incubator for 4 h. At the end of incubation, 25 ul of supernatant were sampled from the cell media to determine the level of lactate dehydrogenase (LDH) released from dead cells. We used an LDH assay adapted from Sigma Co. and followed the experimental procedure according to the manufacture. The LDH activity was determined by monitoring the rate of decrease of NADH absorbency at 340 nM. Background LDH activity was subtracted. The group without drug treatment was used to calculate total cell death due to hydrogen peroxide treatment. The protective effects of PARG inhibitors were expressed as a percentage of cell survival.

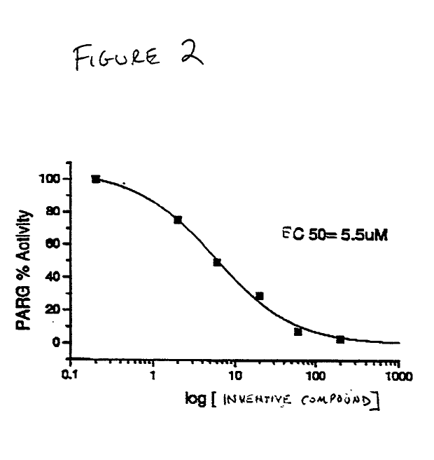

FIG. 2 shows the EC₅₀ that was determined from a cytotoxicity dose responsive curve. To determine the EC₅₀, the concentration of a compound required to achieve 50% reduction of cell death was derived from the dose response curve. Values of percent PARG activity are equivalent to percent reduction in cell death due to a final concentration of 2 mM hydrogen peroxide in the cytotoxicity assay. All methods are the same as described for FIG. 1.

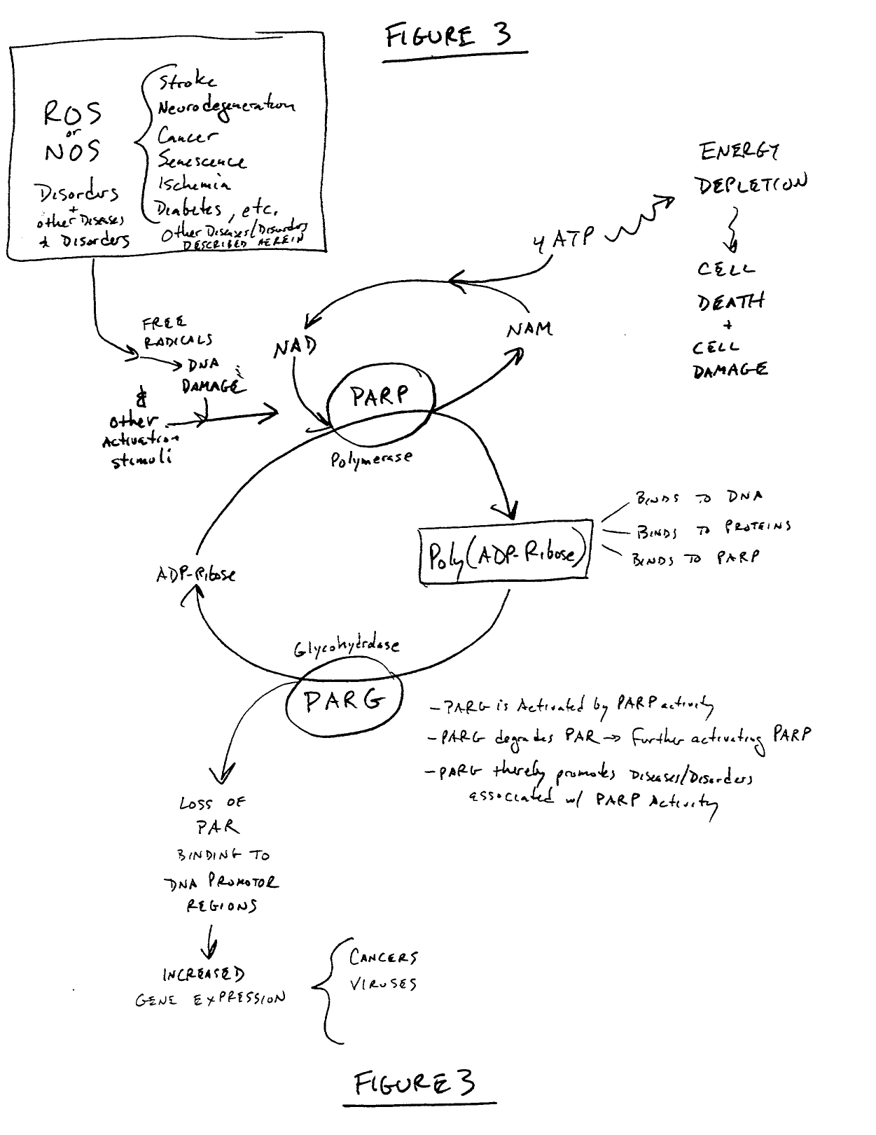

FIG. 3 shows a simplified representation of the PARP/PARG cycle for maintenance of poly(ADP-ribosyl)ation and its relationship to cellular energy metabolism and the various uses, diseases and disorders described herein. The diagram suggests two general mechanisms for how PARG inhibition should be useful for the variety of uses described herein, including for the treatment or prevention of the various diseases and disorders suggested herein. The present invention also contemplates other modes of action for PARG inhibitors not described herein, for the useful methods described herein, such as PARG inhibitors acting on a mechanism of the disease or disorder independent of PAR metabolism. Abbreviations: NAD, nicotinamide adenosine dinucleotide; NAM, nicotinamide; ATP, adenosine triphosphate; ROS, reactive oxygen species; NOS, nitric oxide synthase.

Pharmaceutical Compositions

A further aspect of the present invention is directed to a pharmaceutical composition comprising a pharmaceutically acceptable carrier or a diluent and a therapeutically effective amount of a PARG inhibitor or a pharmaceutically acceptable salt, hydrate, ester, solvate, prodrug, metabolite, or stereoisomer.

PARG inhibitors are useful in the manufacture of pharmaceutical formulations comprising an effective amount thereof in conjunction with or as an admixture with excipients or carriers suitable for either enteral or parenteral application. As such, formulations of the present invention suitable for oral administration may be in the form of discrete units such as capsules, cachets, tablets, troche or lozenges, each containing a predetermined amount of the active ingredient; in the form of a powder or granules; in the form of a solution or a suspension in an aqueous liquid or nonaqueous liquid; or in the form of an oil-in-water emulsion or a water-in-oil emulsion. The active ingredient may also be in the form of a bolus, electuary, or paste.

The composition will usually be formulated into a unit dosage form, such as a tablet, capsule, aqueous suspension or solution. Such formulations typically include a solid, semisolid, or liquid carrier. Exemplary carriers include lactose, dextrose, sucrose, sorbitol, mannitol, starches, gum acacia, calcium phosphate, mineral oil, cocoa butter, oil of theobroma, alginates, tragacanth, gelatin, syrup, methyl cellulose, polyoxyethylene sorbitan monolaurate, methyl hydroxybenzoate, propyl hydroxybenzoate, talc, magnesium stearate, and the like.

Particularly preferred formulations include tablets and gelatin capsules comprising the active ingredient together with (a) diluents, such as lactose, dextrose, sucrose, mannitol, sorbitol, cellulose, dried corn starch, and glycine; and/or (b) lubricants, such as silica, talcum, stearic acid, its magnesium or calcium salt, and polyethylene glycol.

Tablets may also contain binders, such as magnesium aluminum silicate, starch paste, gelatin, tragacanth, methylcellulose, sodium carboxymethylcellulose and polyvinylpyrrolidone; carriers, such as lactose and corn starch; disintegrants, such as starches, agar, alginic acid or its sodium salt, and effervescent mixtures; and/or absorbents, colorants, flavors, and sweeteners. The compositions of the invention may be sterilized and/or contain adjuvants, such as preserving, stabilizing, swelling or emulsifying agents, solution promoters, salts for regulating osmotic pressure, and/or buffers. In addition, the composition may also contain other therapeutically valuable substances. Aqueous suspensions may contain emulsifying and suspending agents combined with the active ingredient. All oral dosage forms may further contain sweetening and/or flavoring and/or coloring agents.

These compositions are prepared according to conventional mixing, granulating, or coating methods, respectively, and contain about 0.1 to 75% of the active ingredient, preferably about 1 to 50% of the same. A tablet may be made by compressing or molding the active ingredient optionally with one or more accessory ingredients. Compressed tablets may be prepared by compressing, in a suitable machine, the active ingredient in a free-flowing form such as a powder or granules, optionally mixed with a binder, lubricant, inert diluent, surface active, or dispersing agent. Molded tablets may be made by molding, in a suitable machine, a mixture of the powdered active ingredient and a suitable carrier moistened with an inert liquid diluent.

When administered parenterally, the composition will normally be in a unit dosage, sterile injectable form (aqueous isotonic solution, suspension or emulsion) with a pharmaceutically acceptable carrier. Such carriers are preferably non-toxic, parenterally-acceptable and contain non-therapeutic diluents or solvents. Examples of such carriers include water; aqueous solutions, such as saline (isotonic sodium chloride solution), Ringer's solution, dextrose solution, and Hanks' solution; and nonaqueous carriers, such as 1,3-butanediol, fixed oils (e.g., corn, cottonseed, peanut, sesame oil, and synthetic mono- or di-glyceride), ethyl oleate, and isopropyl myristate.

Oleaginous suspensions can be formulated according to techniques known in the art using suitable dispersing or wetting agents and suspending agents. Among the acceptable solvents or suspending mediums are sterile fixed oils. For this purpose, any bland fixed oil may be used. Fatty acids, such as oleic acid and its glyceride derivatives, including olive oil and castor oil, especially in their polyoxyethylated forms, are also useful in the preparation of injectables. These oil solutions or suspensions may also contain long-chain alcohol diluents or dispersants.

Sterile saline is a preferred carrier, and the compounds are often sufficiently water soluble to be made up as a solution for all foreseeable needs. The carrier may contain minor amounts of additives, such as substances that enhance solubility, isotonicity, and chemical stability, e.g., antioxidants, buffers and preservatives.

When administered rectally, the composition will usually be formulated into a unit dosage form such as a suppository or cachet. These compositions can be prepared by mixing the compound with suitable non-irritating excipients that are solid at room temperature, but liquid at rectal temperature, such that they will melt in the rectum to release the compound. Common excipients include cocoa butter, beeswax and polyethylene glycols or other fatty emulsions or suspensions.

Moreover, the compounds may be administered topically, especially when the conditions addressed for treatment involve areas or organs readily accessible by topical application, including neurological disorders of the eye, the skin or the lower intestinal tract.

For topical application to the eye, or ophthalmic use, the compounds can be formulated as micronized suspensions in isotonic, pH-adjusted sterile saline or, preferably, as a solution in isotonic, pH-adjusted sterile saline, either with or without a preservative such as benzylalkonium chloride. Alternatively, the compounds may be formulated into ointments, such as petrolatum.

For topical application to the skin, the compounds can be formulated into suitable ointments containing the compounds suspended or dissolved in, for example, mixtures with one or more of the following: mineral oil, liquid petrolatum, white petrolatum, propylene glycol, polyoxyethylene compound, polyoxypropylene compound, emulsifying wax and water. Alternatively, the compounds can be formulated into suitable lotions or creams containing the active compound suspended or dissolved in, for example, a mixture of one or more of the following: mineral oil, sorbitan monostearate, polysorbate 60, cetyl ester wax, cetearyl alcohol, 2-octyldodecanol, benzyl alcohol and water.

Topical application to the lower intestinal tract can be effected in rectal suppository formulations (see above) or in suitable enema formulations.

Formulations suitable for nasal or buccal administration, (such as self-propelling powder dispensing formulations), may comprise about 0.1% to about 5% w/w of the active ingredient or, for example, about 1% w/w of the same. In addition, some formulations can be compounded into a sublingual troche or lozenge.

The formulations may conveniently be presented in unit dosage form and may be prepared by any of the methods well known in the art of pharmacy. All methods include the step of bringing the active ingredient into association with the carrier which constitutes one or more accessory ingredients. In general, the formulations are prepared by uniformly and intimately bringing the active ingredient into association with a liquid carrier or a finely divided solid carrier or both, and then, if necessary, shaping the product into the desired formulation.

In a preferred embodiment, the carrier is a solid biodegradable polymer or mixture of biodegradable polymers with appropriate time release characteristics and release kinetics. The composition of the invention may then be molded into a solid implant suitable for providing efficacious concentrations of the compounds of the invention over a prolonged period of time without the need for frequent redosing. The composition of the present invention can be incorporated into the biodegradable polymer or polymer mixture in any suitable manner known to one of ordinary skill in the art and may form a homogeneous matrix with the biodegradable polymer, or may be encapsulated in some way within the polymer, or may be molded into a solid implant. In one embodiment, the biodegradable polymer or polymer mixture is used to form a soft “depot” containing the pharmaceutical composition of the present invention that can be administered as a flowable liquid, for example, by injection, but which remains sufficiently viscous to maintain the pharmaceutical composition within the localized area around the injection site. The degradation time of the depot so formed can be varied from several days to a few years, depending upon the polymer selected and its molecular wight. By using a polymer composition in injectable form, even the need to make an incision may be eliminated. In any event, a flexible or flowable delivery “depot” will adjust to the shape of the space it occupies within the body with a minimum of trauma to surrounding tissues. The pharmaceutical composition of the present invention is used in amounts that are therapeutically effective and the amounts used may depend upon the desired release profile, the concentration of the pharmaceutical composition required for the sensitizing effect, and the length of time that the pharmaceutical composition has to be released for treatment.

The PARG inhibitors of the invention are preferably administered as a capsule or tablet containing a single or divided dose of the compound, or as a sterile solution, suspension, or emulsion, for parenteral administration in a single or divided dose.

In another preferred embodiment, the PARG inhibitors of the invention can be prepared in lyophilized form. In this case, 1 to 100 mg of a PARG inhibitor may be lyophilized in individual vials, together with a carrier and a buffer, such as mannitol and sodium phosphate. The composition may then be reconstituted in the vials with bacteriostatic water before administration.

The compounds of the invention are used in the composition in amounts that are therapeutically effective. While the effective amount of the PARG inhibitor will depend upon the particular compound being used, amounts of the these compounds varying from about 1% to about 65% have been easily incorporated into liquid or solid carrier delivery systems.

Compositions and Methods for Inhibiting or Decreasing Free-Radical Induced Cellular Energy Depletion, Cell Damage or Cell Death

Preferably, according to the invention, an effective therapeutic amount of the compounds and compositions described above are administered to animals to inhibit or decrease free radical induced cellular energy depletion, cell damage or cell death. In another embodiment of the invention, the pharmaceutical compositions and method of the present invention using PARG inhibitors effect a neuronal activity, that may or may not be mediated by NMDA neurotoxicity or glutamate neurotoxicity. Such neuronal activity may consist of stimulation of damaged neurons, promotion of neuronal regeneration, prevention of neurodegeneration and treatment of a neurological disorder. Accordingly, the present invention further relates to a method of effecting a neuronal activity in an animal, comprising administering an effective amount of the pharmaceutical compositions of the present invention to said animal to treat neural tissue damage, particularly damage resulting from cerebral ischemia and reperfusion injury or neurodegenerative diseases in mammals.

The term “nervous tissue” refers to the various components that make up the nervous system including, without limitation, neurons, neural support cells, glia, Schwann cells, vasculature contained within and supplying these structures, the central nervous system, the brain, the brain stem, the spinal cord, the junction of the central nervous system with the peripheral nervous system, the peripheral nervous system, and allied structures.

The term “neural tissue damage resulting from ischemia and reperfusion injury and neurodegenerative diseases” includes neurotoxicity, such as seen in vascular stroke, global and focal ischemia, and retinal ischemia.

The term “ischemia” refers to localized tissue anemia due to obstruction of the inflow of arterial blood. Global ischemia occurs when blood flow to the entire brain ceases for a period of time. Global ischemia may result from cardiac arrest. Focal ischemia occurs when a portion of the brain is deprived of its normal blood supply. Focal ischemia may result from thromboembolytic occlusion of a cerebral vessel, traumatic head injury, edema or brain tumor. Even if transient, both global and focal ischemia can cause widespread neuronal damage. Although nerve tissue damage occurs over hours or even days following the onset of ischemia, some permanent nerve tissue damage may develop in the initial minutes following the cessation of blood flow to the brain. Much of this damage has been attributed to glutamate toxicity and to the secondary consequences of tissue reperfusion, such as the release of vasoactive products by damaged endothelium and the release of cytotoxic products, such as free radicals and leukotrines, by the damaged tissue. Ischemia can also occur in the heart in myocardial infarction and other cardiovascular disorders in which the coronary arteries have been obstructed as a result of atherosclerosis, thrombi, or spasm.

The term “neurodegenerative diseases” includes Alzheimer's disease, Parkinson's disease and Huntington's disease.

The term “nervous insult” refers to any damage to nervous tissue and any disability or death resulting therefrom. The cause of nervous insult may be metabolic, toxic, neurotoxic, iatrogenic, thermal or chemical, and includes without limitation, ischemia, hypoxia, cerebrovascular accident, trauma, surgery, pressure, mass effect, hemorrhage, radiation, vasospasm, neurodegenerative disease, infection, Parkinson's disease, amyotrophic lateral sclerosis (ALS), myelination/demyelination process, epilepsy, cognitive disorder, glutamate abnormality and secondary effects thereof.

Examples of neurological disorders that are treatable by the method of using the present invention include, without limitation, trigeminal neuralgia; glossopharyngeal neuralgia; Bell's Palsy; myasthenia gravis; muscular dystrophy; amyotrophic lateral sclerosis; progressive muscular atrophy; progressive bulbar inherited muscular atrophy; herniated, ruptured or prolapsed invertebrate disk syndromes; cervical spondylosis; plexus disorders; thoracic outlet destruction syndromes; peripheral neuropathies such as those caused by lead, dapsone, ticks, porphyria, or Guillain-Barré syndrome; Alzheimer's disease; Huntington's Disease and Parkinson's disease.

The method of the present invention is particularly useful for treating a neurological disorder selected from the group consisting of: peripheral neuropathy caused by physical injury or disease state; head trauma, such as traumatic brain injury; physical damage to the spinal cord; stroke associated with brain damage, such as vascular stroke associated with hypoxia and brain damage, focal cerebral ischemia, global cerebral ischemia, and cerebral reperfusion injury; demyelinating diseases, such as multiple sclerosis; and neurological disorders related to neurodegeneration, such as Alzheimer's Disease, Parkinson's Disease, Huntington's Disease and amyotrophic lateral sclerosis (ALS).

The term “neuroprotective” refers to the effect of reducing, arresting or ameliorating nervous insult, and protecting, resuscitating, or reviving nervous tissue that has suffered nervous insult.

The term “preventing neurodegeneration” includes the ability to prevent neurodegeneration in patients diagnosed as having a neurodegenerative disease or who are at risk of developing a neurodegenerative disease. The term also encompasses preventing further neurodegeneration in patients who are already suffering from or have symptoms of a neurodegenerative disease.

The term “treating” refers to:

(i)preventing a disease, disorder or condition from occurring in an animal that may be predisposed to the disease, disorder and/or condition, but has not yet been diagnosed as having it;

(ii)inhibiting the disease, disorder or condition, i.e., arresting its development; and

(iii) relieving the disease, disorder or condition, i.e., causing regression of the disease, disorder and/or condition.

Treating Other PARG-Related Disorders

The compounds, compositions and methods of the invention can also be used to treat a cardiovascular disorder in an animal, by administering an effective amount of the pharmaceutical compositions of the present invention to the animal.

As used herein, the term “cardiovascular disorders” refers to those disorders that can either cause ischemia or are caused by reperfusion of the heart. Examples include, but are not limited to, coronary artery disease, angina pectoris, myocardial infarction, cardiovascular tissue damage caused by cardiac arrest, cardiovascular tissue damage caused by cardiac bypass, cardiogenic shock, and related conditions that would be known by those of ordinary skill in the art or which involve dysfunction of or tissue damage to the heart or vasculature, especially, but not limited to, tissue damage related to PARP activation.

For example, the methods of the invention are believed to be useful for treating cardiac tissue damage, particularly damage resulting from cardiac ischemia or caused by reperfusion injury in mammals. The methods of the invention are particularly useful for treating cardiovascular disorders selected from the group consisting of: coronary artery disease, such as atherosclerosis; angina pectoris; myocardial infarction; myocardial ischemia and cardiac arrest; cardiac bypass; and cardiogenic shock. The methods of the invention are particularly helpful in treating the acute forms of the above cardiovascular disorders.

Further, the methods of the invention can be used to treat tissue damage resulting from cell damage or death due to necrosis or apoptosis, neural tissue damage resulting from ischemia and reperfusion injury, neurological disorders and neurodegenerative diseases; to prevent or treat vascular stroke; to treat or prevent cardiovascular disorders; to treat other conditions and/or disorders such as age-related macular degeneration, immune senescence diseases, arthritis, atherosclerosis, cachexia, degenerative diseases of skeletal muscle involving replicative senescence, diabetes, head trauma, immune senescence, inflammatory bowel disorders (such as colitis and Crohn's disease), muscular dystrophy, osteoarthritis, osteoporosis, pain (such as neuropathic pain), renal failure, retinal ischemia, septic shock (such as endotoxic shock), and skin aging; to extend the lifespan and proliferative capacity of cells; to alter gene expression of senescent cells; or to radiosensitize tumor cells.

The term “treating” refers to:

(i) preventing a disease, disorder or condition from occurring in an animal that may be predisposed to the disease, disorder and/or condition, but has not yet been diagnosed as having it;

(ii) inhibiting the disease, disorder or condition, i.e., arresting its development; and

(iii) relieving the disease, disorder or condition, i.e., causing regression of the disease, disorder and/or condition.

Administration

For medical use, the amount required of a PARG inhibitor to achieve a therapeutic effect will vary according to the particular compound administered, the route of administration, the mammal under treatment, and the particular disorder or disease concerned. A suitable systemic dose of a PARG inhibitor for a mammal suffering from, or likely to suffer from, any condition as described herein is typically in the range of about 0.1 to about 100 mg of base per kilogram of body weight, preferably from about 1 to about 10 mg/kg of mammal body weight. It is understood that the ordinarily skilled physician or veterinarian will readily be able to determine and prescribe the amount of the compound effective for the desired prophylactic or therapeutic treatment.

In so proceeding, the physician or veterinarian may employ an intravenous bolus followed by an intravenous infusion and repeated administrations, as considered appropriate. In the methods of the present invention, the compounds may be administered, for example, orally, parenterally, by inhalation spray, topically, rectally, nasally, buccally, sublingually, vaginally, intraventricularly, or via an implanted reservoir in dosage formulations containing conventional non-toxic pharmaceutically-acceptable carriers, adjuvants and vehicles.

Parenteral includes, but is not limited to, the following examples of administration: intravenous, subcutaneous, intramuscular, intraspinal, intraosseous, intraperitoneal, intrathecal, intraventricular, intrasternal or intracranial injection and infusion techniques, such as by subdural pump. Invasive techniques are preferred, particularly direct administration to damaged neuronal tissue. While it is possible for the PARG inhibitor to be administered alone, it is preferable to provide it as a part of a pharmaceutical formulation.

To be effective therapeutically as central nervous system targets, the compounds used in the methods of the present invention should readily penetrate the blood-brain barrier when peripherally administered. Compounds which cannot penetrate the blood-brain barrier, however, can still be effectively administered by an intraventricular route.

The compounds used in the methods of the present invention may be administered by a single dose, multiple discrete doses or continuous infusion. Since the compounds are small, easily diffusible and relatively stable, they are well suited to continuous infusion. Pump means, particularly subcutaneous or subdural pump means, are preferred for continuous infusion.

For the methods of the present invention, any effective administration regimen regulating the timing and sequence of doses may be used. Doses of the compounds preferably include pharmaceutical dosage units comprising an efficacious quantity of active compound. By an efficacious quantity is meant a quantity sufficient to inhibit PARP activity and/or derive the desired beneficial effects therefrom through administration of one or more of the pharmaceutical dosage units. In a particularly preferred embodiment, the dose is sufficient to prevent or reduce the effects of vascular stroke or other neurodegenerative diseases.

An exemplary daily dosage unit for a vertebrate host comprises an amount of from about 0.001 mg/kg to about 50 mg/kg. Typically, dosage levels on the order of about 0.1 mg to about 10,000 mg of the active ingredient compound are useful in the treatment of the above conditions, with preferred levels being about 0.1 mg to about 1,000 mg. The specific dose level for any particular patient will vary depending upon a variety of factors, including the activity of the specific compound employed; the age, body weight, general health, sex, and diet of the patient; the time of administration; the rate of excretion; any combination of the compound with other drugs; the severity of the particular disease being treated; and the form and route of administration. Typically, in vitro dosage-effect results provide useful guidance on the proper doses for patient administration. Studies in animal models can also be helpful. The considerations for determining the proper dose levels are well-known in the art.

In methods of treating nervous insult (particularly acute ischemic stroke and global ischemia caused by drowning or head trauma), the compounds of the invention can be co-administered with one or more other therapeutic agents, preferably agents which can reduce the risk of stroke (such as aspirin) and, more preferably, agents which can reduce the risk of a second ischemic event (such as ticlopidine).

The compounds and compositions can be co-administered with one or more therapeutic agents either (i) together in a single formulation, or (ii) separately in individual formulations designed for optimal release rates of their respective active agent. Each formulation may contain from about 0.01% to about 99.99% by weight, preferably from about 3.5% to about 60% by weight, of the compound of the invention, as well as one or more pharmaceutical excipients, such as wetting, emulsifying and pH buffering agents. When the compounds used in the methods of the invention are administered in combination with one or more other therapeutic agents, specific dose levels for those agents will depend upon considerations such as those identified above for compositions and methods of the invention in general.

For the methods of the present invention, any administration regimen regulating the timing and sequence of delivery of the compound can be used and repeated as necessary to effect treatment. Such regimen may include pretreatment and/or co-administration with additional therapeutic agents.

To maximize protection of nervous tissue from nervous insult, the compounds of the invention should be administered to the affected cells as soon as possible. In situations where nervous insult is anticipated, the compounds are advantageously administered before the expected nervous insult. Such situations of increased likelihood of nervous insult include surgery, such as carotid endarterectomy, cardiac, vascular, aortic, orthopedic surgery; endovascular procedures, such as arterial catheterization (carotid, vertebral, aortic, cardia, renal, spinal, Adamkiewicz); injections of embolic agents; the use of coils or balloons for hemostasis; interruptions of vascularity for treatment of brain lesions; and predisposing medical conditions such as crescendo transient ischemic attacks, emboli and sequential strokes.

Where pre-treatment for stroke or ischemia is impossible or impracticable, it is important to bring the compounds of the invention into contact with the affected cells as soon as possible, either during or after the event. In the time period between strokes, however, diagnosis and treatment procedures should be minimized to save the cells from further damage and death. Therefore, a particularly advantageous mode of administration with a patient diagnosed with acute multiple vascular strokes is by implantation of a subdural pump to deliver the compound(s) of the invention directly to the infarct area of the brain. Even if comatose, it is expected that the patient would recover more quickly than he or she would without this treatment. Moreover, in any conscious state of the patient, it is expected that any residual neurological symptoms, as well as the re-occurrence of stroke, would be reduced.

As to patients diagnosed with other acute disorders believed to be related to PARP activity, such as diabetes, arthritis and Crohn's disease, the compound of the invention should also be administered as soon as possible in a single or divided dose.

Depending on the patient's presenting symptoms and the degree of response to the initial administration of the compound of the invention, the patient may further receive additional doses of the same or different compounds of the invention, by one of the following routes: parenterally, such as by injection or by intravenous administration; orally, such as by capsule or tablet; by implantation of a biocompatible, biodegradable polymeric matrix delivery system comprising the compound; or by direct administration to the infarct area by insertion of a subdural pump or a central line. It is expected that the treatment would alleviate the disorder, either in part or in its entirety and that fewer further occurrences of the disorder would develop. It also is expected that the patient would suffer fewer residual symptoms.

Where a patient is diagnosed with an acute disorder prior to the availability of the PARG inhibitors of the invention, the patient's condition may deteriorate due to the acute disorder and become a chronic disorder by the time that the PARG inhibitors are available. Even when a patient receives a pharmaceutical composition containing a PARG inhibitor for the chronic disorder, it is also expected that the patient's condition would stabilize and actually improve as a result of receiving the PARG inhibitor.

The PARG inhibitors may also be used for radiosensitizing tumor cells. The term “radiosensitizer”, as used herein, is defined as a molecule, preferably a low molecular weight molecule, administered to animals in therapeutically effective amounts to increase the sensitivity of the cells to be radiosensitized to electromagnetic radiation and/or to promote the treatment of diseases which are treatable with electromagnetic radiation. Diseases which are treatable with electromagnetic radiation include neoplastic diseases, benign and malignant tumors, and cancerous cells. Electromagnetic radiation treatment of other diseases not listed herein are also contemplated by the present invention. The terms “electromagnetic radiation” and “radiation” as used herein includes, but is not limited to, radiation having the wavelength of 10⁻²⁰ to 10⁰ meters. Preferred embodiments of the present invention employ the electromagnetic radiation of: gamma-radiation (10⁻²⁰ to 10⁻¹³ m) x-ray radiation (10⁻¹¹ to 10⁻⁹ m), ultraviolet light (10 nm to 400 nm), visible light (400 nm to 700 nm), infrared radiation (700 nm to 1.0 mm), and microwave radiation (1 mm to 30 cm).

Radiosensitizers are known to increase the sensitivity of cancerous cells to the toxic effects of electromagnetic radiation. Several mechanisms for the mode of action of radiosensitizers have been suggested in the literature including: hypoxic cell radiosensitizers (e.g., 2-nitroimidazole compounds, and benzotriazine dioxide compounds) promote the reoxygenation of hypoxic tissue and/or catalyze the generation of damaging oxygen radicals; non-hypoxic cell radiosensitizers (e.g., halogenated pyrimidines) can be analogs of DNA bases and preferentially incorporate into the DNA of cancer cells and thereby promote the radiation-induced breaking of DNA molecules and/or prevent the normal DNA repair mechanisms; and various other potential mechanisms of action have been hypothesized for radiosensitizers in the treatment of disease.

Many cancer treatment protocols currently employ radiosensitizers activated by the electromagnetic radiation of x-rays. Examples of x-ray activated radiosensitizers include, but are not limited to, the following: metronidazole, misonidazole, desmethylmisonidazole, pimonidazole, etanidazole, nimorazole, mitomycin C, RSU 1069, SR 4233, EO9, RB 6145, nicotinamide, 5-bromodeoxyuridine (BUdR), 5-iododeoxyuridine (IUdR), bromodeoxycytidine, fluorodeoxyuridine (FudR), hydroxyurea, cisplatin, and therapeutically effective analogs and derivatives of the same.

Photodynamic therapy (PDT) of cancers employs visible light as the radiation activator of the sensitizing agent. Examples of photodynamic radiosensitizers include the following, but are not limited to: hematoporphyrin derivatives, Photofrin, benzoporphyrin derivatives, NPe6, tin etioporphyrin SnET2, pheoborbide-a, bacteriochlorophyll-a, naphthalocyanines, phthalocyanines, zinc phthalocyanine, and therapeutically effective analogs and derivatives of the same.

Radiosensitizers may be administered in conjunction with a therapeutically effective amount of one or more other compounds, including but not limited to: compounds which promote the incorporation of radiosensitizers to the target cells; compounds which control the flow of therapeutics, nutrients, and/or oxygen to the target cells; chemotherapeutic agents which act on the tumor with or without additional radiation; or other therapeutically effective compounds for treating cancer or other disease. Examples of additional therapeutic agents that may be used in conjunction with radiosensitizers include, but are not limited to: 5-fluorouracil, leucovorin, 5′-amino-5″deoxythymidine, oxygen, carbogen, red cell transfusions, perfluorocarbons (e.g., Fluosol-DA), 2,3-DPG, BW12C, calcium channel blockers, pentoxyfylline, antiangiogenesis compounds, hydralazine, and L-BSO. Examples of chemotherapeutic agents that may be used in conjunction with radiosensitizers include, but are not limited to: adriamycin, camptothecin, carboplatin, cisplatin, daunorubicin, docetaxel, doxorubicin, interferon (alpha, beta, gamma), interleukin 2, irinotecan, paclitaxel, topotecan, and therapeutically effective analogs and derivatives of the same.

### EXAMPLES

The following examples are illustrative of preferred embodiments of related inventions and are not to be construed as limiting the present invention thereto. All polymer molecular weights are mean average molecular weights. All percentages are based on the percent by weight of the final delivery system or formulation prepared unless otherwise indicated, and all totals equal 100% by weight.

### Example 1

### Assay for Neuroprotective Effects on Focal Cerebral Ischemia in Rats

Focal cerebral ischemia experiments are performed using male Wistar rats weighing 250-300 g, which are anesthetized with 4% halothane. Anesthesia is maintained with 1.0-1.5% halothane until the end of surgery. The animals are installed in a warm environment to avoid a decrease in body temperature during surgery.

An anterior midline cervical incision is made. The right common carotid artery (CCA) is exposed and isolated from the vagus nerve. A silk suture is placed and tied around the CCA in proximity to the heart. The external carotid artery (ECA) is then exposed and ligated with a silk suture. A puncture is made in the CCA and a small catheter (PE 10, Ulrich & Co., St-Gallen, Switzerland) is gently advanced to the lumen of the internal carotid artery (ICA). The pterygopalatine artery is not occluded. The catheter is tied in place with a silk suture. Then, a 4-0 nylon suture (Braun Medical, Crissier, Switzerland) is introduced into the catheter lumen and is pushed until the tip blocks the anterior cerebral artery. The length of catheter into the ICA is approximately 19 mm from the origin of the ECA. The suture is maintained in this position by occlusion of the catheter with heat. One cm of catheter and nylon suture are left protruding so that the suture can be withdrawn to allow reperfusion. The skin incision is then closed with wound clips.

The animals are maintained in a warm environment during recovery from anesthesia. Two hours later, the animals are re-anesthetized, the clips are discarded, and the wound is re-opened. The catheter is cut, and the suture is pulled out. The catheter is then obturated again by heat, and wound clips are placed on the wound. The animals are allowed to survive for 24 hours with free access to food and water. The rats are then sacrificed with CO₂ and decapitated.

The brains are immediately removed, frozen on dry ice and stored at −80° C. The brains are then cut in 0.02 mm-thick sections in a cryocut at −19° C., selecting one of every 20 sections for further examination. The selected sections are stained with cresyl violet according to the Nissl procedure. Each stained section is examined under a light microscope, and the regional infarct area is determined according to the presence of cells with morphological changes.

Various doses of PARG inhibitors are tested in this model. The compounds are administered in either a single dose or a series of multiple doses, i.p. or i.v., at different times, both before or after the onset of ischemia. PARG inhibitors administered in accordance with the methods of the present invention are found to provide protection from ischemia in the range of about 20 to 80%.

### Example 2

### Effects on Heart Ischemia/Reperfusion Injury in Rats

Female Sprague-Dawley rats, each weighing about 300-350 g are anesthetized with intraperitoneal ketamine at a dose of 150 mg/kg. The rats are endotracheally intubated and ventilated with oxygen-enriched room air using a Harvard rodent ventilator. Polyethylene catheters inserted into the carotid artery and the femoral vein are used for artery blood pressure monitoring and fluid administration respectively. Arterial pCO₂ is maintained between 35 and 45 mm Hg by adjusting the respirator rate. The rat chests are opened by median sternotomy, the pericardium is incised, and the hearts are cradled with a latex membrane tent. Hemodynamic data are obtained at baseline after at least a 15-minute stabilization period following the end of the surgical operation. The LAD (left anterior descending) coronary artery is ligated for 40 minutes, and then re-perfused for 120 minutes. After 120 minutes' reperfusion, the LAD artery is re-occluded, and a 0.1 ml bolus of monastral blue dye is injected into the left atrium to determine the ischemic risk region.

The hearts are then arrested with potassium chloride and cut into five 2-3 mm thick transverse slices. Each slice is weighed and incubated in a 1% solution of trimethyltetrazolium chloride to visualize the infarcted myocardium located within the risk region. Infarct size is calculated by summing the values for each left ventricular slice and is further expressed as a fraction of the risk region of the left ventricle.

Various doses of PARG inhibitors are tested in this model. The compounds are given either in a single dose or a series of multiple doses, i.p. or i.v., at different times, both before or after the onset of ischemia. The PARG inhibitors are found to have ischemia/reperfusion injury protection in the range of 10 to 40 percent. Therefore, they protect against ischemia-induced degeneration of rat hippocampal neurons in vitro.

### Example 3

### Retinal Ischemia Protection

A patient just diagnosed with acute retinal ischemia is immediately administered parenterally, either by intermittent or continuous intravenous administration, a PARG inhibitor, either as a single dose or a series of divided doses of the compound. After this initial treatment, and depending on the patient's presenting neurological symptoms, the patient optionally may receive the same or a different PARG inhibitor in the form of another parenteral dose. It is expected by the inventors that significant prevention of neural tissue damage would ensue and that the patient's neurological symptoms would considerably lessen due to the administration of the compound, leaving fewer residual neurological effects post-stroke. In addition, it is expected that the re-occurrence of retinal ischemia would be prevented or reduced.

### Example 4

### Treatment of Retinal Ischemia

A patient has just been diagnosed with acute retinal ischemia. Immediately, a physician or a nurse parenterally administers a PARG inhibitor, either as a single dose or as a series of divided doses. The patient also receives the same or a different PARG inhibitor by intermittent or continuous administration via implantation of a biocompatible, biodegradable polymeric matrix delivery system comprising a PARG inhibitor, or via a subdural pump inserted to administer the compound directly to the infarct area of the brain. It is expected by the inventors that the patient would awaken from the coma more quickly than if the compound of the invention were not administered. The treatment is also expected to reduce the severity of the patient's residual neurological symptoms. In addition, it is expected that re-occurrence of retinal ischemia would be reduced.

### Example 5

### Vascular Stroke Protection

A patient just diagnosed with acute vascular stroke is immediately administered parenterally, either by intermittent or continuous intravenous administration, a PARG inhibitor, either as a single dose or a series of divided doses of the compound. After this initial treatment, and depending on the patient's presenting neurological symptoms, the patient optionally may receive the same or a different compound of the invention in the form of another parenteral dose. It is expected by the inventors that significant prevention of neural tissue damage would ensue and that the patient's neurological symptoms would considerably lessen due to the administration of the compound, leaving fewer residual neurological effects post-stroke. In addition, it is expected that the re-occurrence of vascular stroke would be prevented or reduced.

### Example 6

### Treatment of Vascular Stroke

A patient has just been diagnosed with acute multiple vascular strokes and is comatose. Immediately, a physician or a nurse parenterally administers a PARG inhibitor, either as a single dose or as a series of divided doses. Due to the comatose state of the patient, the patient also receives the same or a different PARG inhibitor by intermittent or continuous administration via implantation of a biocompatible, biodegradable polymeric matrix delivery system comprising a PARG inhibitor, or via a subdural pump inserted to administer the compound directly to the infarct area of the brain. It is expected by the inventors that the patient would awaken from the coma more quickly than if the compound of the invention were not administered. The treatment is also expected to reduce the severity of the patient's residual neurological symptoms. In addition, it is expected that re-occurrence of vascular stroke would be reduced.

### Example 7

### Preventing Cardiac Reperfusion Injury

A patient is diagnosed with life-threatening cardiomyopathy and requires a heart transplant. Until a donor heart is found, the patient is maintained on Extra Corporeal Oxygenation Monitoring (ECMO).

A donor heart is then located, and the patient undergoes a surgical transplant procedure, during which the patient is placed on a heart-lung pump. The patient receives a PARG inhibitor intracardiac within a specified period of time prior to re-routing his or her circulation from the heart-lung pump to his or her new heart, thus preventing cardiac reperfusion injury as the new heart begins to beat independently of the external heart-lung pump.

### Example 8

### Septic Shock Assay

Groups of 10 C57/BL male mice weighing 18 to 20 g are administered a PARG inhibitor at the doses of 60, 20, 6 and 2 mg/kg, daily, by intraperitoneal (IP) injection for three consecutive days. Each animal is first challenged with lipopolysaccharide (LPS, from E. coli, LD₁₀₀ of 20 mg/animal IV) plus galactosamine (20 mg/animal IV). The first dose of test compound in a suitable vehicle is given 30 minutes after challenge, and the second and third doses are given 24 hours later on day 2 and day 3 respectively, with only the surviving animals receiving the second or third dose of the test compound. Mortality was recorded every 12 hours after challenge for the three-day testing period. The PARG inhibitors provide a protection against mortality from septic shock.

### Example 9

### In vitro Radiosensitization

The human prostate cancer cell line, PC-3s, are plated in 6 well dishes and grown at monolayer cultures in RPMI1640 supplemented with 10% FCS. The cells are maintained at 37° C. in 5% CO₂ and 95% air. The cells are exposed to a dose response (0.1 mM to 0.1 μM) of 3 different PARG inhibitors prior to irradiation at one sublethal dose level. For all treatment groups, the six well plates are exposed at room temperature in a Seifert 250 kV/15 mA irradiator with a 0.5 mm Cu/1 mm. Cell viability is examined by exclusion of 0.4% trypan blue. Dye exclusion is assessed visually by microscopy and viable cell number is calculated by subtracting the number of cells from the viable cell number and dividing by the total number of cells. Cell proliferation rates are calculated by the amount of ³H-thymidine incorporation post-irradiation. The PARG inhibitors show radiosensitization of the cells.

### Example 10

### In vivo Radiosensitization

Before undergoing radiation therapy to treat cancer, a patient is administered an effective amount of a pharmaceutical composition containing a PARG inhibitor. The compound or pharmaceutical composition acts as a radiosensitizer and renders the tumor more susceptible to radiation therapy.

### Example 11

### Measuring Altered Gene Expression in mRNA Senescent Cells

Human fibroblast BJ cells, at Population Doubling (PDL) 94, are plated in regular growth medium and then changed to low serum medium to reflect physiological conditions described in Linskens, et al., Nucleic Acids Res. 23:16:3244-3251 (1995). A medium of DMEM/199 supplemented with 0.5% bovine calf serum is used. The cells are treated daily for 13 days with a PARG inhibitor as disclosed herein. The control cells are treated with and without the solvent used to administer the PARG inhibitor. The untreated old and young control cells are tested for comparison. RNA is prepared from the treated and control cells according to the techniques described in PCT Publication No. 96/13610 and Northern blotting is conducted. Probes specific for senescence-related genes are analyzed, and treated and control cells compared. In analyzing the results, the lowest level of gene expression is arbitrarily set at 1 to provide a basis for comparison. Three genes particularly relevant to age-related changes in the skin are collagen, collagenase and elastin. West, Arch. Derm. 130:87-95 (1994). Elastin expression of the cells treated with a PARG inhibitor is significantly increased in comparison with the control cells. Elastin expression is significantly higher in young cells compared to senescent cells, and thus treatment with a PARG inhibitor causes elastin expression levels in senescent cells to change to levels similar to those found in much younger cells. Similarly, a beneficial effect is seen in collagenase and collagen expression with treatment with PARG inhibitors.

### Example 12

### Measuring Altered Gene Expression Protein in Senescent Cells

Approximately 105 BJ cells, at PDL 95-100 are plated and grown in 15 cm dishes. The growth medium is DMEM/199 supplemented with 10% bovice calf serum. The cells are treated daily for 24 hours with a PARG inhibitor (100 μg/1 mL of medium). The cells are washed with phosphate buffered solution (PBS), then permeablized with 4% paraformaldehyde for 5 minutes, then washed with PBS, and treated with 100% cold methanol for 10 minutes. The methanol is removed and the cells are washed with PBS, and then treated with 10% serum to block nonspecific antibody binding. About 1 mL of the appropriate commercially available antibody solutions (1:500 dilution. Vector) is added to the cells and the mixture incubated for 1 hour. The cells are rinsed and washed three times with PBS. A secondary antibody, goat anti-mouse IgG (1 mL) with a biotin tag is added along with 1 mL of a solution containing streptavidin conjugated to alkaline phosphatase and 1 mL of NBT reagent (Vector). The cells are washed and changes in gene expression are noted calorimetrically. Four senescence-specific genes—collagen I, collagen III, collagenase, and interferon gamma—in senescent cells treated with a PARG inhibitor are monitored and the results show a decrease in interferon gamma expression with no observable change in the expression levels of the other three gens, demonstrating that PARG inhibitors can alter senescence-specific gene expression.

### Example 13

### Extending or Increasing Proliferative Capacity and Lifespan of Cells

To demonstrate the effectiveness of the present method for extending the proliferative capacity and lifespan of cells, human fibroblast cells lines (either W138 at Population Doubling (PDL) 23 or BJ cells at PDL 71) are thawed and plated on T75 flasks and allowed to grow in normal medium (DMEM/M199 plus 10% bovine calf serum) for about a week, at which time the cells are confluent, and the cultures are therefor ready to be subdivided. At the time of subdivision, the media is aspirated, and the cells rinsed with phosphate buffer saline (PBS) and then trypsinized. The cells are counted with a Coulter counter and plated at a density of 10⁵ cells per cm² in 6-well tissue culture plates in DMEM/199 medium supplemented with 10% bovine calf serum and varying amounts (0.10 μM, and 1 mM: from a 100×stock solution in DMEM/M199 medium) of a PARG inhibitor. This process is repeated every 7 days until the cell appear to stop dividing. The untreated (control) cells reach senescence and stop dividing after about 40 days in culture. Treatment of cells with 10 μM 3-AB appears to have little or no effect in contrast to treatment with 100 μM 3-AB which appears lengthen the lifespan of the cells and treatment with 1 mM 3-AB which dramatically increases the lifespan and proliferative capacity of the cells. The cells treated with 1 mM 3-AB will still divide after 60 days in culture.

### Example 14

### Neuroprotective Effects of Formula I on Chronic Constriction Injury (CCI) in Rats

Adult male Sprague-Dawley rats, 300-350 g, are anesthetized with intraperitoneal 50 mg/kg sodium pentobarbital. Nerve ligation is performed by exposing one side of the rat's sciatic nerves and dissecting a 5-7 mm-long nerve segment and closing with four loose ligatures at a 1.0-1.5-mm, followed by implanting of an intrathecal catheter and inserting of a gentamicin sulfate-flushed polyethylene (PE-10) tube into the subarachnoid space through an incision at the cisterna magna. The caudal end of the catheter is gently threaded to the lumbar enlargement and the rostral end is secured with dental cement to a screw embedded in the skull and the skin wound is closed with wound clips.

Thermal hyperalgesia to radiant heat is assessed by using a paw-withdrawal test. The rat is placed in a plastic cylinder on a 3-mm thick glass plate with a radiant heat source from a projection bulb placed directly under the plantar surface of the rat's hindpaw. The paw-withdrawal latency is defined as the time elapsed from the onset of radiant heat stimulation to withdrawal of the rat's hindpaw.

Mechanical hyperalgesia is assessed by placing the rat in a cage with a bottom made of perforated metal sheet with many small square holes. Duration of paw-withdrawal is recorded after pricking the mid-plantar surface of the rat's hindpaw with the tip of a safety pin inserted through the cage bottom.

Mechano-allodynia is assessed by placing a rat in a cage similar to the previous test, and applying von Frey filaments in ascending order of bending force ranging from 0.07 to 76 g to the mid-plantar surface of the rat's hindpaw. A von Frey filament is applied perpendicular to the skin and depressed slowly until it bends. A threshold force of response is defined as the first filament in the series to evoke at least one clear paw-withdrawal out of five applications.

Dark neurons are observed bilaterally within the spinal cord dorsal horn, particularly in laminae I-II, of rats 8 days after unilateral sciatic nerve ligation as compared with sham operated rats. Various doses of differing compounds of Formula I are tested in this model and show that the Formula I compounds reduce both incidence of dark neurons and neuropathic pain behavior in CCI rats.

### Example 15

A patient is diagnosed with a disorder requiring the administration of a PARG inhibitor. The patient may then be administered a PARG inhibitor, such as set forth in examples 1 through 10, in the form of a capsule or tablet containing a single or divided dose of the inhibitor. After this initial treatment, the patient may be optionally administered the same or different PARG inhibitor by capsule or tablet, direct injection, subdural pump, or implantation of a biocompatible, polymeric matrix delivery system. It would be expected that the treatment would alleviate the disorder, either in part or in its entirety and that no further occurrences of the disorder would develop.

### Example 16

A treatment such as that described in Example 15 wherein the patient is diagnosed with a peripheral neuropathy caused by physical injury.

### Example 17

A treatment such as that described in Example 15 wherein the patient is diagnosed with a peripheral neuropathy caused by disease state.

### Example 18

A treatment such as that described in Example 15 wherein the patient is diagnosed with Guillain-Barre syndrome.

### Example 19

A treatment such as that described in Example 15 wherein the patient is diagnosed with traumatic brain injury.

### Example 20

A treatment such as that described in Example 15 wherein the patient is diagnosed with physical damage to the spinal cord.

### Example 21

A treatment such as that described in Example 15 wherein the patient is diagnosed with stroke associated with brain damage.

### Example 22

A treatment such as that described in Example 15 wherein the patient is diagnosed with focal ischemia.

### Example 23

A treatment such as that described in Example 15 wherein the patient is diagnosed with global ischemia.

### Example 24

A treatment such as that described in Example 15 wherein the patient is diagnosed with reperfusion injury.

### Example 25

A treatment such as that described in Example 15 wherein the patient is diagnosed with a demyelinating disease.

### Example 26

A treatment such as that described in Example 15 wherein the patient is diagnosed with multiple sclerosis.

### Example 27

A treatment such as that described in Example 15 wherein the patient is diagnosed with a neurological disorder relating to neurodegeneration.

### Example 28

A treatment such as that described in Example 15 wherein the patient is diagnosed with Alzheimer's Disease.

### Example 29

A treatment such as that described in Example 15 wherein the patient is diagnosed with Parkinson's Disease.

### Example 30

A treatment such as that described in Example 15 wherein the patient is diagnosed with amyotrophic lateral sclerosis.

### Example 31

A treatment such as that described in Example 15 wherein the patient is diagnosed with a cardiovascular disease.

### Example 32

A treatment such as that described in Example 15 wherein the patient is diagnosed with angina pectoris.

### Example 33

A treatment such as that described in Example 15 wherein the patient is diagnosed with myocardial infarction.

### Example 34

A treatment such as that described in Example 15 wherein the patient is diagnosed with cardiovascular tissue damage related to PARG activation.

### Example 35

### PARG Enzymatic Assay

The potency of PARG inhibitom was determined in a PARG enzymatic assay. For each compound, various doses were used to inhibit the PARG reaction. A dose responsive curve was generated to determine the IC₅₀ value, the concentration, in uM, required to achieve 50% inhibition of the reaction.

The term “inhibition”, in the context of enzyme inhibition, relates to reversible enzyme inhibition such as competitive, uncompetitive, and noncompetitive inhibition. This can be experimentally distinguished by the effects of the inhibitor on the reaction kinetics of the enzyme, which may be analyzed in terms of the basic Michaelis-Menten rate equation. Competitive inhibition occurs when the inhibitor can combine with the free enzyme in such a way that it competes with the normal substrate for binding at the active site. A competitive inhibitor reacts reversibly with the enzyme to form an enzyme-inhibitor complex [EI], analogous to the enzyme-substrate complex:

E+I==EI

Following the Michaelis-Menten formalism, we can define the inhibitor constant, Kᵢ, as the dissociation constant of the enzyme-inhibitor complex:

$${K}_{i} = \frac{[ E ] [ I ]}{[ EI ]}$$

Thus, in accordance with the above and as used herein, Kᵢ is essentially a measurement of affinity between a molecule, and its receptor, or in relation to the present invention, between the present inventive compounds and the enzyme to be inhibited. It should be noted that IC50 is a related term used when defining the concentration or amount of a compound which is required to cause a 50% inhibition of the target enzyme.

The whole assay consisted of 1) preparation of ³²P-labeled radioactive PARG as substrate, 2) purification of recombinant PARG, 3) incubation of the compound with the PARG reaction, 4) separation of the product ADP-ribose by thin layer chromatography (TL), and 5) quantify the radioactivity of ADP-ribose by scintillation counting.

1). ³²P-poly(ADP-ribose) Preparation:

A 0.1 ml reaction was set up. It consisted of 20 mM TrisHCl (pH 8.0), 10 mM MgCl₂, 5 ug/ml activated DNA (Sigma), 1 uM radioactive NAD (nicotinamide adenine[adenylate-³²P] dinucleotide [³²P]NAD (Amersham) with a specific activity of 100 Ci/mmole). 20 ug/ml of a PARG inhibitor is added last to initiate the reaction. The reaction is mixed thoroughly and incubated at 25° C. for 30 min. The reaction was stopped by the addition of 90 mM EDTA.

At the end of the reaction, ³²P-poly(ADP-ribose) polymer was separated from [³²P]NAD by a sizing column. The 0.1 ml reaction mixture was directly loaded to a prepacked 6 ml sephdax-G25 column (BAKERBOND, Spe, J. T. Baker), which was pre-equilibrated with 1×TE buffer pH 7.5. ³²P-poly(ADP-ribose) was eluted with 1×TE buffer. The elutes were collected in 250 uL fractions. ³²P-poly(ADP-ribose) sample was in an early peak; as determined by scintillation counting.

2). Expression and Purification of Recombinant PARG

A cDNA fragment encoding the carboxyl terminal part of human PARG, from amino acid 378 to 976 was amplified by polymerase chain reaction with human thymus cDNA (Clontech, Palo Alto, Calif.) as template and a pair of primers with the sequences of 5′-GGGAATTCATGAATGATTTAAATGCTAAA-3′ and 5′-CCCTCGAGTCAGGTCCCTGTCCTTTGCCC-3′. The primers contained the restriction enzyme sites EcoRI and XhoI. The PCR amplified PARG DNA fragment was digested with EcoRI and XhoI, and then ligated to the same sites in pGEX-4T1 plasmid (Pharmacia) to create pGEX-PARG by using standard molecular biology procedure. The pGEX-PARG was transformed in to E. coli strain BL21 for expressing the recombinant protein that has a glutathione-S-transferase at the amino terminus and fused in frame with PARG at the carboxy] terminus. We followed the standard procedures for expression and purification of recombinant protein by using the glutathione-sephadex 4B beads according to the manufacture, Pharmacia.

3) PARG Reaction:

A 30 uL reaction was set up. It contained 0.3 ng (200,000 cpm) ³²P-poly(ADP-ribose), the PARG inhibitor, and approximately 0.1 ng/ml PARG. For determine the IC₅₀, a typical experiments consisted compound doses at 0.2, 2, 6, 20, 60 uM final concentrations. Each dose was tested in duplicates. The stock solution of PARG inhibitors were prepared in 100% DMSO. The final concentration of DMSO in the reaction was less than 7%. -The PARG enzyme was added last to initiate the reaction. The reaction was carried on at 37° C. for 10 min. and was then terminated by adding 2 ul of 3% (w/v) sodium dodecyl sulfate.

4). TLC Separation of Hydrolyzed ³²P-ADP-ribose

The whole stopped reaction mixture was carefully spotted on to a 20 cm×20 cm PEI-F cellulose paper (Darmstadt, Germany) at approximately 3 cm from the bottom with 2 cm space between each sample. The PEI-F paper was developed in a TLC tank, pre-equilibrated with 0.3 M LiCl/0.9 M acetic acid in a depth of 2 cm, for 1 h until the developer reached the front of the paper. The PEI-F paper was dried in the air and covered with a plastic wrap and exposed to Kodak X-OMAT film for 3 h.

5). Quantify PARG Activities

The film was developed and used as a template to locate the positions of poly(ADP-ribose) and ADP-ribose on the PEI-F cellulose paper. The upper spot contained ADP-ribose and the lower one contained poly(ADP-ribose). Typically, 10-20% poly(ADP-ribose) was hydrolyzed to ADP-ribose. The corresponding spots were cut out and the radioactivities were determined by scintillation counting. PARG activity was expressed as a percentage of poly(ADP-ribose) converted to ADP-ribose, i.e. the counts of the upper spot divided by the combined total counts of upper and lower spots. A typical dose responsive curve was illustrated in FIG. 1, using a PARG inhibitor in accordance with the present invention.

### Example 36

### Hydrogen Peroxide Cytotoxicity Assay

A hydrogen peroxide cytotoxicity model was used to evaluate the efficacy of a PARG inhibitor to prevent cell death. Poly(ADP-ribose) turn over was shown to be a mechanism that mediated cell death caused by hydrogen peroxide treatment in P338D1 cells, according to Schraustatter et al (Proc. Natl. Acad. Sci. USA, 83, 4908-4912, 1986).

P388D1 cells (ATCC, #CCL-46), derived from murine macrophage like tumor, were maintained in Dulbeco's Modified Eagle Medium (DMEM) with 10% horse serum, 2 mM L-glutamine. The cytotoxicity assay was set up in a 96-well plate. In each well, 190 ul cells were seeded at 2×10⁶/ml density. To determine the EC₅₀, the concentration of a compound required to achieve 50% reduction of cell death, a dose responsive experiment was conducted. Various concentration of a PARG inhibitor was added to the cells. A typical experiment consisted of doses with a final concentrations of 0.01, 0.03, 0.1, 0.3, 1, 3, 10, 30 uM. Each data point was averaged from a quadruplicate. After 15 min incubation, 5 ul of freshly prepared hydrogen peroxide were added to the cells to a final concentration of 2 mM. A set of wells with no compound was not exposed to hydrogen peroxide for background determination. Cells were returned to 37° C. incubator for 4 h. At the end of incubation, 25 ul of supernatant were sampled from the cell media to determine the level of lactate dehydrogenase (LDH) released from dead cells. We used an LDH assay adapted from Sigma Co. and followed the experimental procedure according to the manufacture. The LDH activity was determined by monitoring the rate of decrease of NADH absorbency at 340 nM. Background LDH activity was subtracted. The group without drug treatment was used to calculate total cell death due to hydrogen peroxide treatment. The protective effects of PARG inhibitors were expressed as a percentage of cell survival. The EC₅₀ was determined from a dose responsive curve. As an example, the dose responsive curve for a PARG inhibitor is shown in FIG. 1.

The invention being thus described, it will be obvious that the same may be varied in many ways. Such variations are not to be regarded as a departure from the spirit and scope of the invention and all such modification are intended to be included within the scope of the following claims.

What is claimed is:

1. A pharmaceutical composition comprising a PARG inhibitor or a pharmaceutically acceptable salt, hydrate, ester, solvate, prodrug, metabolite, or stereoisomer thereof, and a pharmaceutically acceptable carrier; wherein the PARG inhibitor is present in an amount that is effective for treatment or prevention of a disease or condition resulting from cell damage or death.

2. The pharmaceutical composition of claim 1 wherein said cell damage or death is due to necrosis, apoptosis, or combinations thereof.

3. The pharmaceutical composition of claim 1 wherein said disease or condition is selected from the group consisting of acute pain, arthritis, atherosclerosis, cachexia, cardiovascular disorders, chronic pain, degenerative diseases, diabetes, diseases or disorders relating to lifespan or proliferative capacity of cells, diseases or disease conditions induced or exacerbated by cellular senescence, head trauma, immune senescence, inflammatory bowel disorders, ischemia, macular degeneration, muscular dystrophy, neural tissue damage resulting from ischemia and reperfusion injury, neurological disorders and neurodegenerative diseases, neuronal mediated tissue damage or disease, neuropathic pain, nervous insult, osteoarthritis, osteoporosis, peripheral nerve injury, renal failure, retinal ischemia, septic shock, skin aging, and vascular stroke.

4. The pharmaceutical composition of claim 3 wherein said PARG inhibitor is selected from the group consisting of glucose derivatives; lignin glycosides; hydrolysable tannins; adenoside derivatives; acridine derivatives; tilorone analogs; daunomycin; ellipticine; and proflavine.

5. The pharmaceutical composition of claim 3 wherein said PARG inhibitor is a compound of formula I: 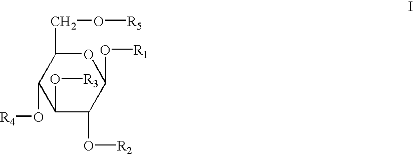
wherein:
R₁, R₂, R₃, R₄, R₅ individually represent a hydrogen atom or X,
X represents a carbonyl having a phenyl individually substituted by a plurality of groups selected from a group consisting of a hydroxyl group and C₁-C₈ alkoxy groups,
provided that R₁-R₅ do not represent a hydrogen atom simultaneously.

6. The pharmaceutical composition of claim 5, wherein X is galloyl, 4-hydroxy-3-methoxybenzoyl, 4-hydroxy-3,5-dimethoxybenzoyl, 3,4,5-trimethoxybenzoyl, 4-hydroxy-3-methoxycinnamoyl, 4-hydroxy-3,5-dimethoxycinnamoyl, 3,4,5-trimethoxycinnamoyl, 3,4,5-trihydroxybenzylcarbonyl or 3,4,5-trihydroxyphenethylcarbonyl.

7. The pharmaceutical composition of claim 6, wherein the compound of formula I is 1,2,3,4,6-penta-o-galloyl-d-glucopyranose, 1,2,3,4,6-penta-o-(3,5-dimethoxy-4-hydroxycinnamoyl)-d-glucopyranose, or 1,2,3,4,6-penta-o-(3,4,5-trimethoxycinnamoyl)-d-glucopyranose.

8. The pharmaceutical composition of claim 3, wherein said PARG inhibitor is a hydrolysable tannin.

9. The pharmaceutical composition of claim 8, wherein said hydrolysable tannin is selected from the group consisting of gallotannins and ellagitannins.

10. The pharmaceutical composition of claim 3, wherein said PARG inhibitor is a lignin glycoside.

11. The pharmaceutical composition of claim 10, wherein said lignin glycoside has the following properties:
(i) tannin and polysaccharide are bonded;
(ii) the molecular weight is 500 to 140,000;
(iii) the bonding ratio of tannin to polysaccharide is 1:1 to 20:1, as a molecular ratio;
(iv) the polysaccharide is composed of 60 to 70% uronic acid, and 30 to 40% neutral sugar.

12. The pharmaceutical composition of claim 11, wherein the lignin glycoside comprises the following structure:

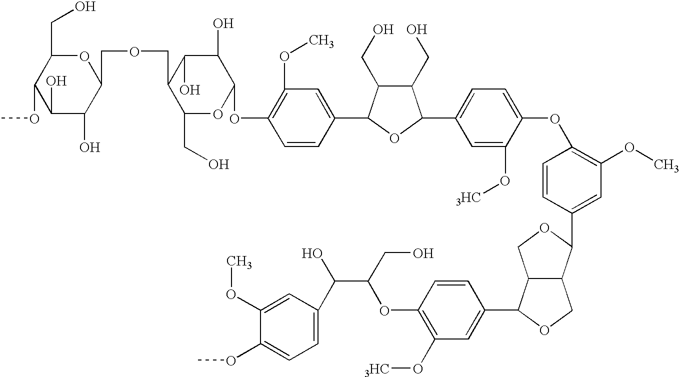

13. The pharmaceutical composition of claim 3, wherein said PARG inhibitor comprises a compound of formula II: 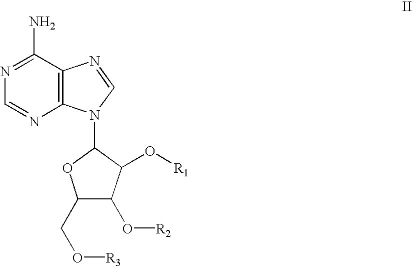
wherein:
R₁ represents a hydrogen atom, a group represented by formula III: 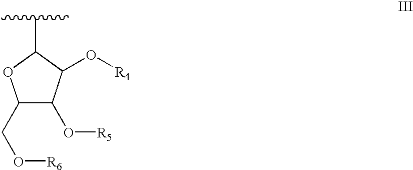
or X, wherein X is the compound of formula IV: 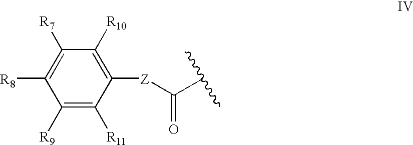
wherein Z is a bond, C₁-C₈ alkyl, or C₂-C₈ alkenyl;
R₇, R₈, R₉, R₁₀, and R₁₁ are independently selected from hydrogen, hydroxyl, or C₁-C₈ alkoxy, provided that R₇-R₁₁ are not four or five hydrogen atoms simultaneously, and R₂, R₃, R₄, R₅, and R₆ independently represent a hydrogen atom or X, X representing the same as that described above;
provided that R₁, R₂, and R₃ do not represent a hydrogen atom simultaneously;
and further provided that R₂, R₃, R₄, R₅, and R₆ do not represent a hydrogen atom simultaneously.

14. The pharmaceutical composition of claim 13, wherein X is galloyl, 4-hydroxy-3-methoxybenzoyl, 4-hydroxy-3,5-dimethoxybenzoyl, 3,4,5-trimethoxybenzoyl, 4-hydroxy-3-methoxycinnamoyl, 4-hydroxy-3,5-dimethoxycinnamoyl, 3,4,5-trimethoxycinnamoyl, 3,4,5-trihydroxybenzylcarbonyl or 3,4,5-trihydroxyphenethylcarbonyl.

15. A pharmaceutical composition comprising a PARG inhibitor or a pharmaceutically acceptable salt, hydrate, ester, solvate, prodrug, metabolite, or stereoisomer thereof, and a pharmaceutically acceptable carrier; wherein the PARG inhibitor is present in an amount that is effective for inhibiting or decreasing free radical-induced cellular energy depletion.

16. A pharmaceutical composition comprising a compound of formula I: 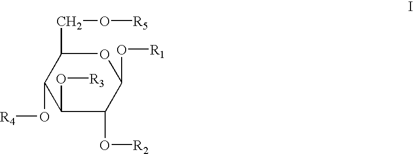
or a pharmaceutically acceptable salt, hydrate, ester, solvate, prodrug, metabolite, or stereoisomer thereof, and a pharmaceutically acceptable carrier;
wherein the compound of formula I is present in an amount that is effective for treatment or prevention of diseases or conditions selected from the group consisting of tissue damage resulting from cell damage or death due to necrosis or apoptosis, neuronal mediated tissue damage or diseases, neural tissue damage resulting from ischemia and reperfusion injury, neurological disorders and neurodegenerative diseases, vascular stroke, cardiovascular disorders, macular degeneration, arthritis, atherosclerosis, cachexia, degenerative diseases of skeletal muscle, diabetes, head trauma, inflammatory bowel disorders, muscular dystrophy, osteoarthritis, osteoporosis, neuropathic pain, nervous insult, peripheral nerve injury, renal failure, retinal ischemia, septic shock, and skin aging;
and wherein:
R₁, R₂, R₃, R₄, R₅ individually represent a hydrogen atom or A, A representing a carbonyl having a phenyl substituted by a plurality of groups selected from a group consisting of a hydroxyl group and C₁-C₈ alkoxy groups, provided that R₁-R₅ do not represent a hydrogen atom simultaneously.

17. The pharmaceutical composition of claim 16, wherein A is galloyl, 4-hydroxy-3-methoxybenzoyl, 4-hydroxy-3, 5-dimethoxybenzoyl, 3,4,5-trimethoxybenzoyl, 4-hydroxy-3-methoxycinnamoyl, 4-hydroxy-3,5-dimethoxycinnamoyl, 3,4,5-trimethoxycinnamoyl, 3,4,5-trihydroxybenzylcarbonyl or 3,4,5-trihydroxyphenethylcarbonyl.

18. The pharmaceutical composition of claim 16, wherein the compound of formula I is 1,2,3,4,6-penta-o-galloyl-d-glucopyranose, 1,2,3,4,6-penta-o-(3,5-dimethoxy-4-hydroxycinnamoyl)-d-glucopyranose, or 1,2,3,4,6-penta-o-(3,4,5-trimethoxycinnamoyl)-d-glucopyranose.

19. A pharmaceutical composition comprising a compound of formula I: 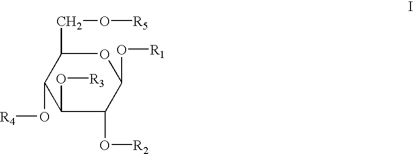
or a pharmaceutically acceptable salt, hydrate, ester, solvate, prodrug, metabolite, or stereoisomer thereof, and a pharmaceutically acceptable carrier;
wherein the compound of formula I is present in an amount that is effective for inhibiting or decreasing free radical-induced cellular energy depletion;
and wherein:
R₁, R₂, R₃, R₄, R₅ individually represent a hydrogen atom or A, A representing a carbonyl having a phenyl substituted by a plurality of groups selected from a group consisting of a hydroxyl group and C₁-C₈ alkoxy groups, provided that R₁-R₅ do not represent a hydrogen atom simultaneously.

20. A pharmaceutical composition comprising a lignin glycoside or a pharmaceutically acceptable salt, hydrate, ester, solvate, prodrug, metabolite, or stereoisomer thereof, and a pharmaceutically acceptable carrier;
wherein the lignin glycoside is present in an amount that is effective for treatment or prevention of diseases or conditions selected from the group consisting of tissue damage resulting from cell damage or death due to necrosis or apoptosis, neuronal mediated tissue damage or diseases, neural tissue damage resulting from ischemia and reperfusion injury, neurological disorders and neurodegenerative diseases, vascular stroke, cardiovascular disorders, macular degeneration, arthritis, atherosclerosis, cachexia, degenerative diseases of skeletal, diabetes, head trauma, inflammatory bowel disorders, muscular dystrophy, osteoarthritis, osteoporosis, neuropathic pain, nervous insult, peripheral nerve injury, renal failure, retinal ischemia, septic shock, and skin aging;
and wherein:
the lignin glycoside has the following properties:
(i) tannin and polysaccharide are bonded;
(ii) the molecular weight is 500 to 140,000;
(iii) the bonding ratio of tannin to polysaccharide is 1:1 to 20:1, as a molecular ratio;
(iv) the polysaccharide is composed of 60 to 70% uronic acid, and 30 to 40% neutral sugar.

21. The pharmaceutical composition of claim 20, wherein the lignin glycoside comprises the following structure:

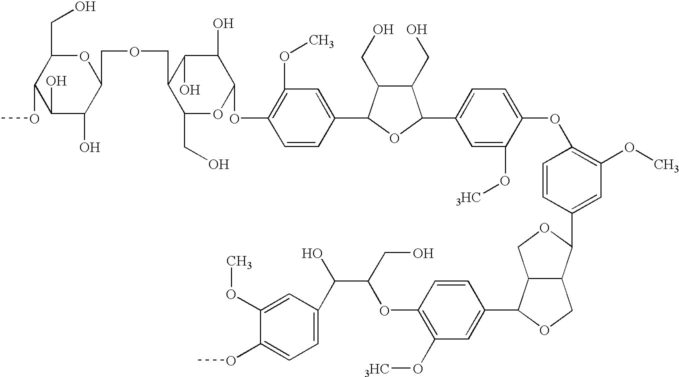

22. A pharmaceutical composition comprising a lignin glycoside or a pharmaceutically acceptable salt, hydrate, ester, solvate, prodrug, metabolite, or stereoisomer thereof, and a pharmaceutically acceptable carrier;
wherein the lignin glycoside is present in an amount that is effective for inhibiting or decreasing free radical-induced cellular energy depletion; and wherein:
the lignin glycoside has the following properties:
(i) tannin and polysaccharide are bonded;
(ii) the molecular weight is 500 to 140,000;
(iii) the bonding ratio of tannin to polysaccharide is 1:1 to 20:1, as a molecular ratio; and
(iv) the polysaccharide is composed of 60 to 70% uronic acid, and 30 to 40% neutral sugar.

23 A pharmaceutical composition comprising a compound of formula II: 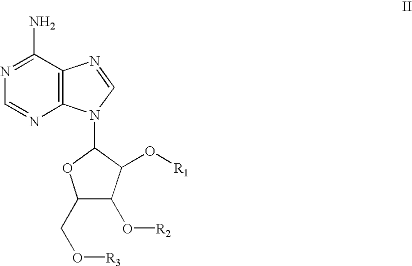
or a pharmaceutically acceptable salt, hydrate, ester, solvate, prodrug, metabolite, or stereoisomer thereof, and a pharmaceutically acceptable carrier;
wherein the compound of formula II is present in an amount that is effective for treatment or prevention of diseases or conditions selected from the group consisting of tissue damage resulting from cell damage or death due to necrosis or apoptosis, neuronal mediated tissue damage or diseases, neural tissue damage resulting from ischemia and reperfusion injury, neurological disorders and neurodegenerative diseases, vascular stroke, cardiovascular disorders, macular degeneration, arthritis, atherosclerosis, cachexia, degenerative diseases of skeletal muscle, diabetes, head trauma, inflammatory bowel disorders, muscular dystrophy, osteoarthritis, osteoporosis, neuropathic pain, nervous insult, peripheral nerve injury, renal failure, retinal ischemia, septic shock, and skin aging; and wherein:
R₁ represents a hydrogen atom, a group represented by formula III: 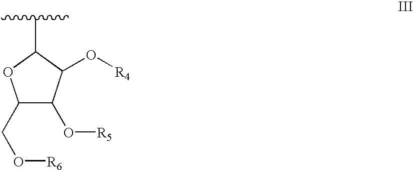
or A, wherein A is the compound of formula IV: 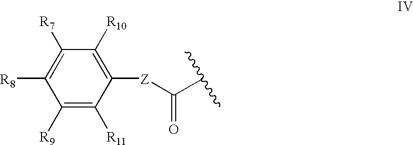
wherein Z is a bond, C₁-C₈ alkyl, or C₂-C₈ alkenyl;
R₇, R₈, R₉, R₁₀ and R₁₁ are independently selected from hydrogen, hydroxyl, or C₁-C₈ alkoxy, provided that R₇-R₁₁ are not four or five hydrogen atoms simultaneously, and R₂, R₃, R₄, R₅, and R6 independently represent a hydrogen atom or A, A representing the same as that described above;
provided that R₁, R₂, and R₃ do not represent a hydrogen atom simultaneously; and further provided that R₂, R₃, R₄, R₅, and R₆ do not represent a hydrogen atom simultaneously.

24. The pharmaceutical composition of claim 23, wherein A is galloyl, 4-hydroxy-3-methoxybenzoyl, 4-hydroxy-3,5-dimethoxybenzoyl, 3,4,5-trimethoxybenzoyl, 4-hydroxy-3-methoxycinnamoyl, 4-hydroxy-3,5-dimethoxycinnamoyl, 3,4,5-trimethoxycinnamoyl, 3,4,5-trihydroxybenzylcarbonyl or 3,4,5-trihydroxyphenethylcarbonyl.

25. A pharmaceutical composition comprising a compound of formula II: 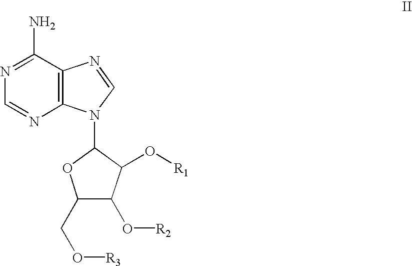
or a pharmaceutically acceptable salt, hydrate, ester, solvate, prodrug, metabolite, or stereoisomer thereof, and a pharmaceutically acceptable carrier;
wherein the compound of formula II is present in an amount that is effective for inhibiting or decreasing free radical-induced cellular energy depletion;
and wherein:
R₁ represents a hydrogen atom, a group represented by formula III: 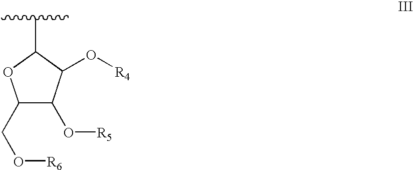
or A, wherein A is the compound of formula IV: 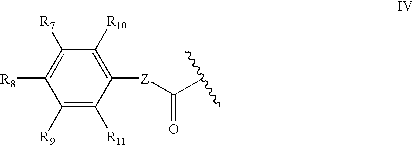
wherein Z is a bond, C₁-C₈ alkyl, or C₂-C₈ alkenyl;
R₇, R₈, R₉, R₁₀, and R₁₁ are independently selected from hydrogen, hydroxyl, or C₁-C₈ alkoxy, provided that R₇-R₁₁ are not four or five hydrogen atoms simultaneously, and R₂, R₃, R₄, R₅, and R₆ independently represent a hydrogen atom or A, A representing the same as that described above;
provided that R₁, R₂, and R₃ do not represent a hydrogen atom simultaneously;
and further provided that R₂, R₃, R₄, R₅, and R₆ do not represent a hydrogen atom simultaneously.

26. A method for inhibiting or decreasing free radical-induced cellular energy depletion comprising administering to an animal a therapeutically effective amount of a PARG inhibitor or a pharmaceutically acceptable salt, hydrate, ester, solvate, prodrug, metabolite, or stereoisomer thereof.

27. A method for inhibiting or preventing free radical-induced cell death or cell damage comprising administering to an animal a therapeutically effective amount of a PARG inhibitor or a pharmaceutically acceptable salt, hydrate, ester, solvate, prodrug, metabolite, or stereoisomer thereof.

28. A method for treating or preventing diseases or conditions selected from the group consisting of tissue damage resulting from cell damage or death due to necrosis or apoptosis, neuronal mediated tissue damage or diseases, neural tissue damage resulting from ischemia and reperfusion injury, neurological disorders and neurodegenerative diseases, vascular stroke, cardiovascular disorders, macular degeneration, arthritis, atherosclerosis, cachexia, degenerative diseases of skeletal muscle, diabetes, head trauma, inflammatory bowel disorders, muscular dystrophy, osteoarthritis, osteoporosis, neuropathic pain, nervous insult, peripheral nerve injury, radiosensitizing of tumor cells, renal failure, retinal ischemia, septic shock, and skin aging; comprising administering to an animal a therapeutically effective amount of a compound of formula I: 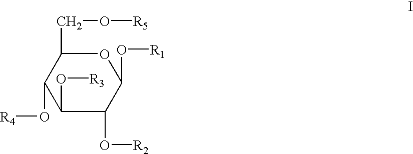
or a pharmaceutically acceptable salt, hydrate, ester, solvate, prodrug, metabolite, or stereoisomer thereof;
wherein:
R₁, R₂, R₃, R₄, R₅ individually represent a hydrogen atom or A, A representing a carbonyl having a phenyl substituted by a one or more substituents of groups selected from a group consisting of a hydroxyl group and C₁-C₈ alkoxy groups, provided that R₁-R₅ do not represent a hydrogen atom simultaneously.

29. The method of claim 28, wherein A is galloyl, 4-hydroxy-3-methoxybenzoyl, 4-hydroxy-3,5-dimethoxybenzoyl, 3,4,5-trimethoxybenzoyl, 4-hydroxy-3-methoxycinnamoyl, 4-hydroxy-3,5-dimethoxycinnamoyl, 3,4,5-trimethoxycinnamoyl, 3,4,5-trihydroxybenzylcarbonyl or 3,4,5-trihydroxyphenethylcarbonyl.

30. The method of claim 28, wherein the compound of formula I is 1,2,3,4,6-Penta-O-Galloyl-D-Glucopyranose, 1,2,3,4,6-Penta-O-(3,5-Dimethoxy-4-Hydroxycinnamoyl)-D-Glucopyranose, or 1,2,3,4,6-Penta-O-(3,4,5-Trimethoxycinnamoyl)-D-Glucopyranose.

31. A method for inhibiting or decreasing free radical-induced cellular energy depletion comprising administering to an animal a therapeutically effective amount of a compound of formula I: 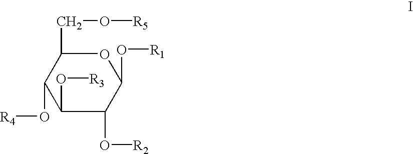
or a pharmaceutically acceptable salt, hydrate, ester, solvate, prodrug, metabolite, or stereoisomer thereof;
and wherein:
R₁, R₂, R₃, R₄, R₅ individually represent a hydrogen atom or A, A representing a carbonyl having a phenyl substituted by a plurality of groups selected from a group consisting of a hydroxyl group and C₁-C₈ alkoxy groups, provided that R₁-R₅ do not represent a hydrogen atom simultaneously.

32. A method for treating or preventing diseases or conditions selected from the group consisting of tissue damage resulting from cell damage or death due to necrosis or apoptosis, neuronal mediated tissue damage or diseases, neural tissue damage resulting from ischemia and reperfusion injury, neurological disorders and neurodegenerative diseases, vascular stroke, cardiovascular disorders, macular degeneration, arthritis, atherosclerosis, cachexia, degenerative diseases of skeletal muscle, diabetes, head trauma, inflammatory bowel disorders, muscular dystrophy, osteoarthritis, osteoporosis, neuropathic pain, nervous insult, peripheral nerve injury, renal failure, retinal ischemia, septic shock, and skin aging; comprising administering to an animal a therapeutically effective amount of a lignin glycoside or a pharmaceutically acceptable salt, hydrate, ester, solvate, prodrug, metabolite, or stereoisomer thereof; wherein:
the lignin glycoside has the following properties:
(i) tannin and polysaccharide are bonded;
(ii) the molecular weight is 500 to 140,000;
(iii) the bonding ratio of tannin to polysaccharide is 1:1 to 20:1, as a molecular ratio;
(iv) the polysaccharide is composed of 60 to 70% uronic acid, and 30 to 40% neutral sugar.

33. The pharmaceutical composition of claim 32, wherein the tannin glycoside comprises the following structure:

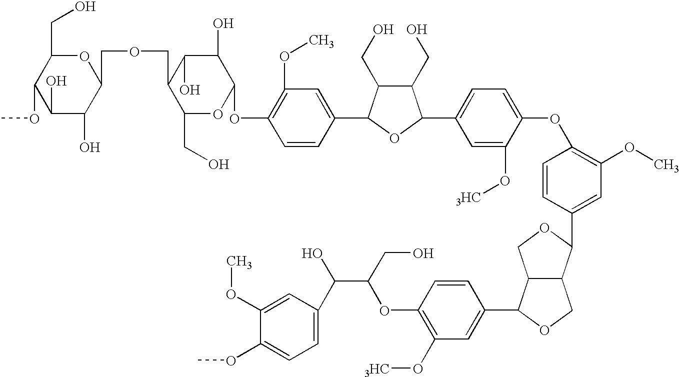

34. A method for inhibiting or decreasing free radical-induced cellular energy depletion comprising administering to an animal a therapeutically effective amount of a lignin glycoside or a pharmaceutically acceptable salt, hydrate, ester, solvate, prodrug, metabolite, or stereoisomer thereof;
wherein:
the lignin glycoside has the following properties:
(i) tannin and polysaccharide are bonded;
(ii) the molecular weight is 500 to 140,000;
(iii) the bonding ratio of tannin to polysaccharide is 1:1 to 20:1, as a molecular ratio; and
(iv) the polysaccharide is composed of 60 to 70% uronic acid, and 30 to 40% neutral sugar.

35. A method for treating or preventing diseases or conditions selected from the group consisting of tissue damage resulting from cell damage or death due to necrosis or apoptosis, neuronal mediated tissue damage or diseases, neural tissue damage resulting from ischemia and reperfusion injury, neurological disorders and neurodegenerative diseases, vascular stroke, cardiovascular disorders, macular degeneration, arthritis, atherosclerosis, cachexia, degenerative diseases of skeletal muscle, diabetes, head trauma, inflammatory bowel disorders, muscular dystrophy, osteoarthritis, osteoporosis, neuropathic pain, nervous insult, peripheral nerve injury, renal failure, retinal ischemia, septic shock, and skin aging;
comprising administering to an animal a therapeutically effective amount of a compound of formula II: 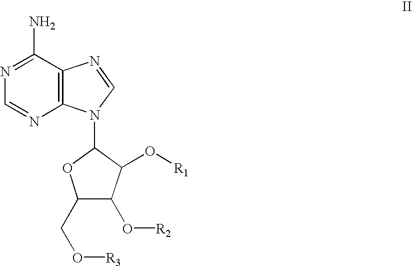
or a pharmaceutically acceptable salt, hydrate, ester, solvate, prodrug, metabolite, or stereoisomer thereof;
wherein:
R₁ represents a hydrogen atom, a group represented by formula III: 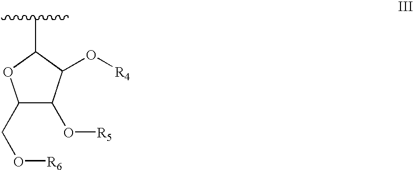
or A, A representing a carbonyl having a phenyl substituted by a plurality of groups selected from a group consisting of a hydroxyl group and C₁-C₈ alkoxy groups, and R₂, R₃, R₄, R₅, and R₆ independently represent a hydrogen atom or A, A representing the same as that described above;
provided that R₁, R₂, and R₃ do not represent a hydrogen atom simultaneously; and further provided that R₂, R₃, R₄, R₅, and R₆ do not represent a hydrogen atom simultaneously.

37. The method of claim 36, wherein A is galloyl, 4-hydroxy-3-methoxybenzoyl, 4-hydroxy-3,5-dimethoxybenzoyl, 3,4,5-trimethoxybenzoyl, 4-hydroxy-3-methoxycinnamoyl, 4-hydroxy-3,5-dimethoxycinnamoyl, 3,4,5-trimethoxycinnamoyl, 3,4,5-trihydroxybenzylcarbonyl or 3,4,5-trihydroxyphenethylcarbonyl.

38. A method for inhibiting or decreasing free radical-induced cellular energy depletion comprising administering to an animal a therapeutically effective amount of a compound of formula II: 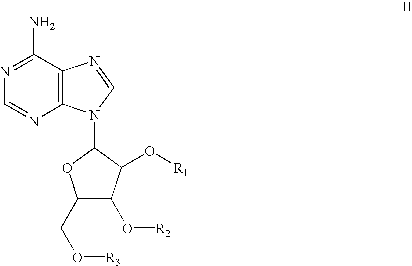
or a pharmaceutically acceptable salt, hydrate, ester, solvate, prodrug, metabolite, or stereoisomer thereof;
wherein:
R₁ represents a hydrogen atom, a group represented by formula III: 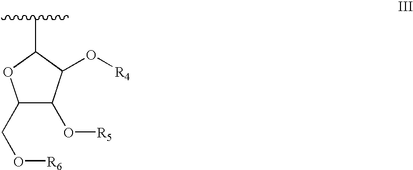
or A, A representing a carbonyl having a phenyl substituted by a plurality of groups selected from a group consisting of a hydroxyl group and C₁-C₈ alkoxy groups, and R₂, R₃, R₄, R₅, and R₆ independently represent a hydrogen atom or A, A representing the same as that described above; provided that R₁, R₂, and R₃ do not represent a hydrogen atom simultaneously;
and further provided that R₂, R₃, R₄, R₅, and R₆ do not represent a hydrogen atom simultaneously.

39. A pharmaceutical composition comprising a PARG inhibitor or a pharmaceutically acceptable salt, hydrate, ester, solvate, prodrug, metabolite, or stereoisomer thereof, and a pharmaceutically acceptable carrier; wherein the PARG inhibitor is present in an amount that is effective for radiosensitizing tumor cells.
